SEQUENCE LISTING (10 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 160 aa, Homo sapiens
  synthetic peptide
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 6 aa, Artificial Sequence
  synthetic peptide
HHHHHH

SEQ ID NO: 4 - protein, 8 aa, Artificial Sequence
  synthetic peptide
MKHHHHHH

SEQ ID NO: 5 - protein, 10 aa, Artificial Sequence
  synthetic peptide
MKHHAHHQHH

SEQ ID NO: 6 - protein, 14 aa, Artificial Sequence
  synthetic peptide
MKHQHQHQHQHQHQ

SEQ ID NO: 7 - protein, 15 aa, Artificial Sequence
  synthetic peptide
MKHQHQHQHQHQHQQ

SEQ ID NO: 8 - protein, 10 aa, Artificial Sequence
  synthetic peptide
EQKLISEEDL

SEQ ID NO: 9 - protein, 8 aa, Artificial Sequence
  synthetic peptide
DYKDDDDK

SEQ ID NO: 10 - protein, 9 aa, Artificial Sequence
  synthetic peptide
YPYDVPDYA

(19) United States
(12) Patent Application Publication
(10) Pub. No.: US 2003/0186386 A1
(43) Pub. Date: Oct. 2, 2003

(54) Interleukin 10

(76) Inventors: Christian Karsten Hansen, Vedbaek (DK); Kim Vilbour Andersen, Broenshoej (DK)

Correspondence Address: MAXYGEN, INC., INTELLECTUAL PROPERTY DEPARTMENT, 515 GALVESTON DRIVE, RED WOOD CITY, CA, 94063, US

(21) Appl. No.: 10/203,531

(22) PCT Filed: Feb. 9, 2001
(86) PCT No.: PCT/DK01/00091

(30) Foreign Application Priority Data
Feb. 11, 2000 (DK) PA 2000 00221
Sep. 5, 2000 (DK) PA 2000 01325

(51) Int. Cl. A61K 38/20; C07K 14/52; C07H 21/04; C12P 21/04

(52) U.S. Cl. 435/69.52; 435/320.1; 435/325; 530/351; 424/85.2; 536/23.5

(57) ABSTRACT

Conjugates exhibiting IL-10 activity comprising (i) a polypeptide which comprises an amino acid sequence that differs from that of human IL-10 in at least one amino acid residue selected from an introduced or removed amino acid residue comprising an attachment group for the non-polypeptide moiety of (ii), and (ii) a non-polypeptide moiety. The conjugates have increased half-life as compared to hIL-10 and may be used for treatment of, e.g., inflammatory diseases.

### FIELD OF THE INVENTION

The present invention relates to new polypeptides exhibiting interleukin 10 (IL-10) activity, to conjugates between a polypeptide exhibiting IL-10 activity and a non-polypeptide moiety, to methods of preparing such polypeptides or conjugates, and the use of such polypeptides or conjugates in therapy, in particular for the treatment of inflammatory diseases such as rheumatoid arthritis.

### BACKGROUND OF THE INVENTION

IL-10 was initially described as an activity in the supernatants of activated T-helper type 2 (Th2) clones that could inhibit the production of cytokines, especially interferon gamma (IFN-gamma) by T-helper type 1 (Th1) clones (J Exp Med 1989; 170:2081-2095). Viral homologues of IL-10 have been detected in the genomes of Epstein-Barr virus (EBV) and equine herpesvirus 2 (Science 1990; 248:1230-1234) (Proc Natl Acad Sci USA 1991; 88:1172-1176) (Virus Genes 1993; 7:111-116).

IL-10 has also been described under the names cytokine synthesis inhibitory factor (CSIF), mast cell growth factor III (MCGF-III) and B-cell derived T-cell growth factor (B-TCGF).

Mature human IL-10 (hIL-10) consists of 160 amino acid residues, is biologically active as a homodimer and is derived from a precursor consisting of 178 amino acid residues. These sequences are reported herein as SEQ ID No 2 and SEQ ID No 1, respectively. The DNA sequences encoding these proteins have been reported (Human Cytokines, Handbook of Basic and Clinical Research, Volume II, Blackwell Science, Eds. Aggarwal and Gutterman, 1996, pp. 19-42). The same publication discloses the IL-10 receptor, its amino acid sequence and underlying DNA sequences, a bioassay for IL-10, methods of purifying IL-10, as well as further information of relevance to IL-10.

Il-10 has two intramolecular disulphide bonds and an un-occupied glycosylation site. The three-dimensional structure of IL-10 has been reported (Biochemistry 1995; 34:12118-12125) (Structure 1995; 3:591-601) (Protein Sci. 1996 5:1955-1962).

IL-10 has been suggested as an anti-inflammatory agent for treatment of inflammatory bowel disease, rheumatoid arthritis, uveitis, etc. It has also been suggested in connection with transplantation, immunodeficiencies and parasitic infections.

Currently, a recombinant human IL-10 (rhIL-10)-based product is in clinical trials. One of the draw-backs of therapy with recombinant human proteins, including IL-10, is the need for frequent administration by injection, as well as a potential for development of neutralizing antibodies in response to therapy with such molecules.

Therefore, a clinical need exists for a product that provides part or all of the therapeutically relevant effects of IL-10, and which may be administered at less frequent intervals and/or which has a reduced immunogenic potential as compared to rhIL-10. The present invention is directed to such compositions of matter as well as the means of making such.

### BRIEF DISCLOSURE OF THE INVENTION

More specifically, the present invention relates to polypeptide conjugates exhibiting IL-10 activity and methods for their preparation and their use in medical treatment.

Accordingly, in its first aspect the invention relates to a conjugate exhibiting IL-10 activity comprising

i) a polypeptide which comprises an amino acid sequence that differ from the amino acid sequence shown in SEQ ID NO 2 in at least one amino acid residue selected from an introduced or removed amino acid residue comprising an attachment group for the non-polypeptide moiety of ii), and ii) a non-polypeptide moiety.

In a further aspect the invention relates to a generally novel polypeptide exhibiting IL-10 activity, which polypeptide forms part of a conjugate of the invention. The polypeptide of the invention is contemplated to be useful as such for therapeutic, diagnostic or other purposes, but find particular interest as an intermediate product for the preparation of a conjugate of the invention.

In a still further aspect the invention relates to a substantially homogenous preparation of a conjugate of the invention.

In still further aspects the invention relates to means and methods for preparing a conjugate or a polypeptide of the invention, including nucleotide sequences and expression vectors encoding a polypeptide or a conjugate of the invention.

In final aspects the invention relates to a therapeutic composition comprising a conjugate, polypeptide or preparation of the invention and methods of treating a mammal with such composition. In particular the polypeptide, conjugate or composition of the invention may be used to treat inflammatory diseases, such as rheumatoid arthritis, and in connection with transplantation, immunodeficiencies and parasitic infections.

### DETAILED DISCLOSURE OF THE INVENTION

Definitions

In the context of the present application and invention the following definitions apply:

The term “conjugate” is intended to indicate a heterogeneous (in the sense of composite or chimeric) molecule formed by the covalent attachment of one or more polypeptide(s) to one or more non-polypeptide moieties such as polymer molecules, lipophilic compounds, carbohydrate moieties or organic derivatizing agents. The term covalent attachment means that the polypeptide and the non-polypeptide moiety are either directly covalently joined to one another, or else are indirectly covalently joined to one another through an intervening moiety or moieties, such as a bridge, spacer, or linkage moiety or moieties. Preferably, the conjugate is soluble at relevant concentrations and conditions, i.e. soluble in physiological fluids such as blood. Examples of conjugated polypeptides of the invention include glycosylated and/or PEGylated polypeptides. The term “non-conjugated polypeptide” may be used about the polypeptide part of the conjugate.

The “polymer molecule” is a molecule formed by covalent linkage of two or more monomers, wherein none of the monomers is an amino acid residue, except where the polymer is human albumin or another abundant plasma protein. The term “polymer” may be used interchangeably with the term “polymer molecule”. The term is intended to cover carbohydrate molecules attached by in vitro glycosylation. Carbohydrate molecules attached by in vivo glycolsylation, such as N- or O-glycosylation (as further described below)) are referred to herein as “a sugar moiety”. Except where the number of non-polypeptide moieties, such as polymer molecule(s) or sugar moieties in the conjugate is expressly indicated every reference to “a non-polypeptide moiety” contained in a conjugate or otherwise used in the present invention shall be a reference to one or more non-polypeptide moieties, such as polymer molecule(s) or sugar moieties, in the conjugate.

The term “attachment group” is intended to indicate a functional group of the polypeptide, in particular of an amino acid residue thereof or a carbohydrate moiety, capable of attaching a non-polypeptide moiety such as a polymer molecule, a lipophilic molecule, a sugar moiety or an organic derivatizing agent. Useful attachment groups and their matching non-peptide moieties are apparent from the table below.

| | | Examples of | Conjugation | |
|---|---|---|---|---|
| Attachment | Amino | non-peptide | method/- | |
| group | acid | moiety | Activated PEG | Reference |
| —NH₂ | N-terminal, | Polymer, e.g. | mPEG-SPA | Shearwater Inc. |
| | Lys | PEG, with amide | Tresylated | Delgado et al, critical |
| | | or imine group | mPEG | reviews in Therapeu- |
| | | | | tic Drug Carrier Sys- |
| | | | | tems 9(3,4): 249-304 |
| | | | | (1992) |
| —COOR | C-term, | Polymer, e.g. | mPEG-Hz | Shearwater Inc. |
| | Asp, Glu | PEG, with ester | | |
| | | or amide group | | |
| | | Carbohydrate | In vitro coupling | |
| | | moiety | | |
| —SH | Cys | Polymer, e.g. | PEG- | Shearwater Inc. |
| | | PEG, with disul- | vinylsulphone | Delgado et al, critical |
| | | fide, maleimide | PEG-maleimide | reviews in Therapeu- |
| | | or vinyl sulfone | | tic Drug Carrier Sys- |
| | | group | | tems 9(3,4): 249-304 |
| | | | In vitro coupling | (1992) |
| | | Carbohydrate | | |
| | | moiety | | |
| —OH | Ser, Thr, | Sugar moiety | In vivo O-linked | |
| | OH—, Lys | | glycosylation | |
| | | PEG with ester, | | |
| | | ether, carbamate, | | |
| | | carbonate | | |
| —CONH₂ | Asn as part | Sugar moiety | In vivo N- | |
| | of an N- | | glycosylation | |
| | glycosyla- | Polymer, e.g. | | |
| | tion site | PEG | | |
| Aromatic | Phe, Tyr, | Carbohydrate | In vitro coupling | |
| residue | Trp | moiety | | |
| —CONH₂ | Gln | Carbohydrate | In vitro coupling | Yan and Wold, Bio- |
| | | moiety | | chemistry, 1984, Jul |
| | | | | 31; 23(16): 3759-65 |
| Aldehyde | Oxidized | Polymer, e.g. | PEGylation | Andresz et al., 1978, |
| Ketone | oligo- | PEG, | | Makromol. Chem. |
| | saccharide | PEG-hydrazide | | 179: 301, WO |
| | | | | 92/16555, WO |
| | | | | 00/23114 |
| Guanidino | Arg | Carbohydrate | In vitro coupling | Lundblad and Noyes, |
| | | moiety | | Chimical Reagents |
| | | | | for Protein Modifica- |
| | | | | tion, CRC Press Inc., |
| | | | | Florida, USA |
| Imidazole | His | Carbohydrate | In vitro coupling | As for guanidine |
| ring | | moiety | | |

For in vivo N-glycosylation, the term “attachment group” is used in an unconventional way to indicate the amino acid residues constituting an N-glycosylation site (with the sequence N-X′-S/T/C-X″, wherein X′ is any amino acid residue except proline, X″ any amino acid residue that may or may not be identical to X′ and preferably is different from proline, N is asparagine and S/T/C is either serine, threonine or cysteine, preferably serine or threonine, and most preferably threonine). Although the asparagine residue of the N-glycosylation site is the one to which the sugar moiety is attached during glycosylation, such attachment cannot be achieved unless the other amino acid residues of the N-glycosylation site is present. Accordingly, when the non-polypeptide moiety is an N-linked sugar moiety, the term “amino acid residue comprising an attachment group for the non-polypeptide moiety” as used in connection with alterations of the amino acid sequence of the parent polypeptide is to be understood as amino acid residues constituting an N-glycosylation site is/are to be altered in such a manner that either a functional N-glycosylation site is introduced into the amino acid sequence or removed from said sequence.

In the present application, amino acid names and atom names (e.g. CA, CB, CD, CG, SG, NZ, N, O, C, etc) are used as defined by the Protein DataBank (PDB) (www.pdb.org) based on the IUPAC nomenclature (IUPAC Nomenclature and Symbolism for Amino Acids and Peptides (residue names, atom names e.t.c.), Eur. J. Biochiem., 138, 9-37 (1984) together with their corrections in Eur. J. Biochem., 152, 1 (1985). The term “amino acid residue” is intended to indicate an amino acid residue contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The terminology used for identifying amino acid positions/substitutions is illustrated as follows: T13 indicates position #13 occupied by a threonine residue in the amino acid sequence shown in SEQ ID NO 2. T13K indicates that the threonine residue of position 13 has been substituted with a lysine residue. The numbering of amino acid residues made herein is made relative to the amino acid sequence shown in SEQ ID NO 2. Multiple substitutions are indicated with a “+”, e.g. S93N+G95S/T means an amino acid sequence which comprises a substitution of the serine residue in position 93 with an asparagine residue and a substitution of the glycine residue in position 95 with a serine or a threonine residue.

The term “nucleotide sequence” is intended to indicate a consecutive stretch of two or more nucleotide molecules. The nucleotide sequence may be of genomic, cDNA, RNA, semisynthetic, synthetic origin, or any combinations thereof.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

“Cell”, “host cell”, “cell line” and “cell culture” are used interchangeably herein and all such terms should be understood to include progeny resulting from growth or culturing of a cell. “Transformation” and “transfection” are used interchangeably to refer to the process of introducing DNA into a cell.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

The term “introduce” is primarily intended to mean substitution of an existing amino acid residue, but may also mean insertion of an additional amino acid residue. The term “remove” is primarily intended to mean substitution of the amino acid residue to be removed by another amino acid residue, but may also mean deletion (without substitution) of the amino acid residue to be removed.

The term “immunogenicity” as used in connection with a given substance is intended to indicate the ability of the polypeptide or conjugate to induce a response from the immune system. The immune response may be a cell or antibody mediated response (see, e.g., Roitt: Essential Immunology (8^{th} Edition, Blackwell) for further definition of immunogenicity). Normally reduced antibody reactivity will be an indication of a reduced immunogenicity.

The term “functional in vivo half-life” is used in its normal meaning, i.e the time at which 50% of the biological activity of the polypeptide or conjugate is still present in the body/target organ, or the time at which the activity of the polypeptide or conjugate is 50% of the initial value. As an alternative to determining functional in vivo half-life, “serum half-life” may be determined, i.e. the time in which 50% of the polypeptide or conjugate molecules circulate in the plasma or bloodstream prior to being cleared. Determination of serum half-life is often more simple than determining the functional in vivo half-life and the magnitude of serum half-life is usually a good indication of the magnitude of functional in vivo half-life. Alternatively terms to serum half-life include “plasma half-life”, “circulating half-life”, “serum clearance”, “plasma clearance” and “clearance half-life”. The polypeptide or conjugate is cleared by the action of one or more of the reticuloendothelial systems (RES), kidney, spleen or liver, by IL-10-receptormediated degradation, or by specific or unspecific proteolysis. Normally, clearance depends on size (relative to the cutoff for glomerular filtration), charge, attached carbohydrate chains, and the presence of cellular receptors for the protein. The functionality to be retained is normally selected from proliferative or receptor binding activity. The functional in vivo half-life and the serum half-life may be determined by any suitable method known in the art as further discussed in the Methods section hereinafter.

The term “increased” as used about the functional in vivo half-life or serum half-life is used to indicate that the relevant half-life of the conjugate or polypeptide is statistically significantly increased relative to that of a reference molecule, such as an unconjugated hIL-10 (e.g. Tenovil®) as determined under comparable conditions.

The term “renal clearance” is used in its normal meaning to indicate any clearance taking place by the kidneys, e.g. by glomerular filtration, tubular excretion or tubular elimination. Renal clearance depends on physical characteristics of the conjugate, including molecular weight, size (diameter), hydrodynamic volume, symmetry, shape/rigidity, and charge. Usually, a molecular weight of about 67 kDa is considered to be a cut-off-value for renal clearance. Reduced renal clearance may be established by any suitable assay, e.g. an established in vivo assay. Typically, renal clearance is determined by administering a labelled (e.g. radiolabelled or fluorescence labelled) polypeptide conjugate to a patient and measuring the label activity in urine collected from the patient. Reduced renal clearance is determined relative to the corresponding non-conjugated polypeptide or the non-conjugated corresponding wild-type polypeptide under comparable conditions.

The term “exhibiting IL-10 activity” is intended to indicate that the polypeptide or conjugate has one or more of the functions of native IL-10, in particular hIL-10 with the amino acid sequence shown in SEQ ID NO 2, including the capability to bind to a IL-10 receptor. The IL-10 activity is conveniently assayed using the primary assay described in the Materials and Methods section hereinafter. The polypeptide or conjugate “exhibiting” IL-10 activity is considered to have such activity, when it displays a measurable function, e.g. a measurable proliferative activity (e.g. as determined by the primary assay described in the Materials and Methods section) or a receptor binding activity which can be determined using assays known in the art. Normally, a “measurable function” is an in vitro bioactivity of at least 2%, such as at least 10%. The polypeptide exhibiting IL-10 activity may also be termed “IL-10 molecule” herein.

The term “parent IL-10” or “parent polypeptide” is intended to indicate the molecule to be modified in accordance with the present invention. The parent IL-10 is normally hIL-10 or a variant thereof, but may also be of a non-human, preferably mammalian origin. A “variant” is a polypeptide, which differs in one or more amino acid residues from a parent polypeptide, normally in 1, 2, 3, 4, 5, 6, 7, 8, 9, 10, 11, 12, 13, 14 or 15 amino acid residues. An example of rhIL-10 is Tenovil®.

The present invention is illustrated using hIL-10 as a parent polypeptide. However, in case another parent polypeptide is used it will be understood that this will be modified in a position that is equivalent to a position indicated herein for modification of hIL-10. An “equivalent position” is intended to indicate a position in the amino acid sequence of the relevant parent polypeptide, which is homologous (i.e. corresponding in position in either primary or tertiary structure) to a position in the amino acid sequence shown in SEQ ID NO 2. The “equivalent position” is conveniently determined on the basis of an alignment of the relevant polypeptide sequences, e.g. using the program CLUSTALW version 1.74 using default parameters (Thompson et al., 1994, CLUSTAL W: improving the sensitivity of progressive multiple sequence alignment through sequence weighting, position-specific gap penalties and weight matrix choice, Nucleic Acids Research, 22:4673-4680) or from published alignments.

Conjugate of the Invention

As stated above, in a first aspect the invention relates to a conjugate exhibiting IL-10 activity comprising i) a polypeptide which comprises an amino acid sequence that differs from the amino acid sequence of SEQ ID NO 2 in at least one amino acid residue selected from an introduced or removed amino acid residue comprising an attachment group for the non-polypeptide moiety of ii), and ii) a non-polypeptide moiety. The amino acid residues to be introduced and/or removed are described in further detail in the following sections.

The conjugate of the invention is the result of a generally new strategy for developing improved molecules with IL-10 activity. More specifically, by removing and/or introducing an amino acid residue comprising an attachment group for the non-polypeptide moiety it is possible to specifically adapt the polypeptide so as to make the molecule more susceptible to conjugation to the non-polypeptide moiety of choice, to optimize the conjugation pattern (e.g. to ensure an optimal distribution of non-polypeptide moieties on the surface of the IL-10 molecule and to ensure that only the attachment groups intended to be conjugated is present in the molecule) and thereby obtain a new conjugate molecule, which has IL-10 activity and in addition one or more improved properties as compared to IL-10 molecules available today.

While the polypeptide i) may be of any origin, in particular mammalian origin, it is presently preferred to be of human origin. Normally, the polypeptide i) exhibits IL-10 activity. Furthermore, the polypeptide is preferably in its homodimeric form. Preferably, the conjugate comprises a sufficient number of non-polypeptide moieties to render the conjugate less susceptible to renal clearance than hIL-10. Normally, the number of non-polypeptides required for this purpose is determined by the molecular weight of the non-polypeptide moiety.

In a preferred embodiment one difference between the amino acid sequence of the polypeptide i) and the amino acid sequence shown in SEQ ID NO 2 is that at least one and preferably more, e.g. 1-15, amino acid residues comprising an attachment group for the non-polypeptide moiety ii) has been introduced, preferably by substitution, into the amino acid sequence. Thereby, for instance, shielding by non-polypeptide moieties may be achieved in greater or different regions of the polypeptide molecule leading to a lower immune response, and/or the molecular weight, shape, size and/or charge of the conjugate optimised. Preferably, such amino acid residue is introduced in a position occupied by an amino acid residue having more than 25%, such as more than 50% or even more than 75% of its side chain exposed at the surface of the molecule. Such positions are identified in the Materials and Methods section herein.

The term “one difference” as used in the present application is intended to allow for additional differences being present. Accordingly, in addition to the specified amino acid difference (to introduce and/or remove an attachment group), the polypeptide may contain additional amino acid changes as compared to SEQ ID NO 2, which changes may have been introduced for any purpose.

In a further preferred embodiment one difference between the amino acid sequence of the polypeptide i) and the amino acid sequence shown in SEQ ID NO 2 is that at least one and preferably more, e.g. 1-15, amino acid residues comprising an attachment group for the non-polypeptide moiety ii) has/have been removed, preferably by substitution, from the amino acid sequence. The amino acid residue to be removed is preferably one to which conjugation is disadvantageous, e.g. an amino acid residue located at or near a functional site of the polypeptide (since conjugation at such a site may result in inactivation or reduced IL-10 activity of the resulting conjugate due to impaired receptor recognition). In the present context the term “functional site” is intended to indicate one or more amino acid residues which is/are essential for or otherwise involved in the function or performance of hIL-10, in particular receptor binding and/or activation and residues essential for dimerization of the polypeptide. Such amino acid residues are a part of the functional site. The functional site may be determined by methods known in the art and is preferably identified by analysis of a structure of the polypeptide complexed to a relevant receptor, such as the hIL-10 receptor. In a still further preferred embodiment, the amino acid sequence of the polypeptide i) differs from the amino acid sequence shown in SEQ ID NO 2 in that a) at least one amino acid residue comprising an attachment group for the non-polypeptide moiety and present in the amino acid sequence shown in SEQ ID NO 2 has been removed, preferably by substitution, and b) at least one amino acid residue comprising an attachment group for the non-polypeptide moiety has been introduced into the amino acid sequence, preferably by substitution. For instance, the amino acid residue(s) may be any of those described in the subsequent sections herein. This embodiment is considered of particular interest in that it is possible to specifically design the polypeptide i) so as to obtain an optimal conjugation to the non-polypeptide moiety of choice. For instance, by introducing and removing selected amino acid residues as exemplified in the following sections it is possible to ensure an optimal distribution of attachment groups for the non-polypeptide moiety of choice, which gives rise to a conjugate in which the non-polypeptide moieties are placed so as to effectively shield epitopes and other surface parts of the polypeptide without substantially impairing the function of the polypeptide i).

In preferred embodiments of the present invention more than one amino acid residue of the polypeptide with IL-10 activity is altered, e.g. the alteration embraces removal as well as introduction of amino acid residues comprising an attachment group for the non-polypeptide moiety of choice. In addition to the removal and/or introduction of amino acid residues the polypeptide i) may comprise other substitutions or glycosylations which are not related to introduction and/or removal of amino acid residues comprising an attachment group for the non-polypeptide moiety.

Normally, the total number of amino acid residues to be altered in accordance with the present invention, e.g. as described in the subsequent sections herein, (as compared to the amino acid sequence shown in SEQ ID NO 2) does not exceed 15. The exact number of amino acid residues and the type of amino acid residues to be introduced depend, i.a., on the desired nature and degree of conjugation (e.g. the identity of the non-polypeptide moiety, how many non-polypeptide moieties it is desirable or possible to conjugate to the polypeptide, where they should be conjugated, etc.). The exact number of amino acid residues and the type of amino acid residues to be removed depend, i.a., on the desired nature and degree of conjugation (e.g. the identity of the non-polypeptide moiety, whether and where in the polypeptide conjugation should be avoided, etc.). Preferably, the polypeptide part of the conjugate of the invention or the polypeptide of the invention comprises an amino acid sequence, which differs in 1-15 amino acid residues from the amino acid sequence shown in SEQ ID NO 2, such as in 1-8 or 2-8 amino acid residues, e.g. in 1-5 or 2-5 amino acid residue from the amino acid sequence shown in SEQ ID NO 2. Thus, normally the polypeptide part of the conjugate or the polypeptide of the invention comprises an amino acid sequence which differs from the amino acid sequence shown in SEQ ID NO 2 in 1, 2, 3, 4, 5, 6, 7, 8, 9, 10, 11, 12, 13, 14 or 15 amino acid residues.

The amino acid residue comprising an attachment group for a non-polypeptide moiety, either it be removed or introduced, is selected on the basis of the nature of the non-polypeptide moiety of choice and, in most instances, on the basis of the method in which conjugation between the polypeptide i) and the non-polypeptide moeity ii) is to be achieved. It will be understood that in order to preserve a measurable function of the conjugate or polypeptide, amino acid residues to be modified (by deletion, preferably by substitution) are selected from those amino acid residues that are not essential for providing a measurable activity. Accordingly, amino acid residues to be modified are different from those required for subunit dimerization and/or receptor binding or activation. The identity of such amino acid residues are described in the prior art (a representative part of which is identified in the Background section above) or can be determined by a person skilled in the art using methods known in the art (e.g. using available 3 D structure of IL-10 complexed to its receptor).

In the conjugate of the invention it is preferred that at least 50% of all conjugatable attachment groups, such as at least about 80% and preferably all of such groups are occupied by the relevant non-polypeptide moiety. Accordingly, in a preferred embodiment the conjugate of the invention comprises, e.g., 1-10 non-polypeptide moieties, such as 2-8 or 3-6 non-polypeptide moieties.

Preferably, the conjugate of the present invention has one or more improved properties as compared to hIL-10, in particular rhIL-10 (e.g. Tenovil®), including increased functional in vivo half-life, increased serum half-life, reduced renal clearance, reduced immunogenicity and/or increased bioavailability. Consequently, medical treatment with a conjugate of the invention offers a number of advantages over the currently available IL-10 compounds, including longer duration between injections and fewer side effects.

Normally, the increased functional in vivo half-life is obtained as a consequence of the conjugate having a reduced susceptibility to renal clearance as compared to hIL-10 or Tenovil®. The reduced susceptibility to renal clearance is obtained as a consequence of the molecular weight, size, shape/rigidity, net charge and other characteristics of the conjugate being changed as compared to the unconjugated polypeptide. Typically, the conjugate according to the invention has a molecular weight of at least 67 kDa, preferably at least 70 kDa, although also a lower molecular weight may give rise to a reduced renal clearance. Polymer molecules, such as PEG, have been found to be particularly useful for adjusting the molecular weight of the conjugate.

Preferably, the conjugate of the invention has a reduced renal clearance of at least 50%, preferably by at least 75%, and most preferably with at least 90% as compared to the corresponding non-conjugated polypeptide or the corresponding non-conjugated wild-type polypeptide (such as hIL-10 or rhIL-10) as determined under comparable conditions.

Preferably, the functional in vivo half-life is increased by at least a factor of two, in particular at least a factor of 5.

The polypeptide i) is preferably any of the polypeptides disclosed in the subsequent sections having introduced and/or removed amino acid residues comprising an attachment group for the non-polypeptide moiety ii).

Conjugate of the Invention, Wherein the Non-Polypeptide Moiety is Attached to a lysine or the N-Terminal Amino Acid Residue

In a preferred embodiment the conjugate of the invention is one, wherein the amino acid residue comprising an attachment group for the non-polypeptide moiety is a lysine residue and the non-polypeptide moiety ii) is any molecule which has lysine as an attachment group. For instance, the non-polypeptide moiety is a polymer molecule, in particular any of the molecules mentioned in the section entitled “Conjugation to a polymer molecule”, and preferably selected from the group consisting of linear or branched polyethylene glycol or polyalkylene oxide. Most preferably, the polymer molecule is PEG and the activated molecule to be used for conjugation is SS-PEG, NPC-PEG, aldehyd-PEG, MPEG-SPA, mPEG-SCM, MPEG-BTC from Shearwater Polymers, Inc, SC-PEG from Enzon, Inc., tresylated PEG as described in U.S. Pat. No. 5,880,255, or oxycarbonyl-oxy-N-dicarboxyimide-PEG (U.S. Pat. No. 5,122,614). Normally, for conjugation to a lysine residue the non-polypeptide moiety has a molecular weight of about 5 or 10 kDa.

The polypeptide i) having introduced and/or removed at least one lysine is preferably in vivo glycosylated, e.g. using naturally-occurring glycosylation sites present in the polypeptide. However, in a particular embodiment the conjugate is one, wherein the amino acid sequence of the polypeptide i) differ(s) from that of SEQ ID NO 2 in that an N-glycosylation site has been introduced and/or removed. Such introduced/removed site(s) may be any of those described in the section entitled “Conjugate of the invention wherein the non-polypeptide moiety is a sugar moiety”.

i) Removal of Lysine Residue(s)

hIL-10 contains thirteen lysine residues one or more of which may be located in the receptor-binding domain. In order to avoid conjugation to one or more of these lysine residues (since this may inactivate or severely reduce the activity of the resulting conjugate) it may be desirable to remove at least one lysine residue. Accordingly, the conjugate according to this embodiment comprises

i) a polypeptide comprising an amino acid sequence that differs from the amino acid sequence shown in SEQ ID NO 2 in in the removal of at least one lysine residue selected from the group consisting of K34, K40, K49, K57, K88, K99, K117, K119, K125, K130, K134, K138 and K157, in particular selected from the group consisting of K34, K40, K49, K57, K88, K99, K117, K119, K125, K130, K134, and K157 (having more than 25% of their side chains surface exposed, even more preferably from the group consisting of K34, K49, K57, K88, K99, K117, K119, and K130 (having more than 50% of their side

chain surface exposed), and ii) a non-polypeptide moiety, which has a lysine residue as an attachment group.

The removal is preferably achieved by substitution with any other amino acid residue, in particular an arginine or a glutamine residue.

ii) Introduction of Lysine Residue(s)

In order to obtain a more extensive or differently distributed conjugation it is desirable to introduce at least one non-naturally occurring lysine residue in hIL-10, in particular in a position which in hIL-10 is occupied by an amino acid residue having a side chain which is more than 25% surface exposed and not part of a cystine or located at a receptor binding site. Such amino acid residues are identified in the Examples section hereinafter or form part of the state of the art.

Accordingly, in a further embodiment the conjugate of the invention is one, which comprises a non-polypeptide moiety having lysine as attachment group and a polypeptide i) comprising an amino acid sequence that differs from that shown in SEQ ID NO 2 in the introduction of at least one lysine residue in a position selected from the group consisting of S1, P2, G3, Q4, G5, T6, Q7, S8, E9, N10, S11, T13, H14, P16, G17, N18, P20, N21, R24, D25, R27, D28, S31, R32, T35, Q38, M39, Q42, L43, D44, N45, L46, E50, S51, E54, G58, S66, Q70, E74, E75, P78, Q79, N82, Q83, D84, P85, D86, A89, H90, N92, S93, E96, N97, T100, R102, L103, R106, R107, H109, R 110, P113, E122, Q123, N126, A127, N129, Q132, E133, D144,I145, N148, E151, T155, M156, R159 and N160 (having more than 25% surface exposed side chain), more preferably from the group consisting of S1, P2, G3, Q4, G5, T6, Q7, S8, E9, N10, S11, T13, H14, P16, G17, P20, N21, R24, D25, D28, S31, R32, T35, Q38, M39, Q42, D44, N45, E50, E54, G58, Q70, E74, Q79, N82, P85, D86, A89, H90, S93, E96, T100, L103, R107, H109, E122, N126, A127, N129, Q132, E133, D144, N148, T155, R159 and N160 (residues having more than 50% surface exposed side chain), and most preferably from the group consisting of R24, R27, R32, R102, R106, R107, R110 and R159, even more preferably R24, R32, R107 and R159.

The introduction of a lysine residue is preferably achieved by substitution of any of the above amino acid residues.

iii) Introduction and Removal of Lysine Residue(s)

In a preferred embodiment the conjugate of the invention comprises at least one introduced lysine residue, in particular any of those described in the section entitled “Introduction of lysine residue(s)”, and at least one removed lysine residue, in particular any of those described in the section entitled “Removal of lysine residue(s)”.

For instance, the conjugate comprises a polypeptide i) comprising an amino acid sequence that differs from the amino acid sequence shown in SEQ ID NO 2 in that at least one lysine residue has been introduced, by substitution, in a position selected from the group consisting of S1, P2, G3, Q4, G5, T6, Q7, S8, E9, N10, S11, T13, H14, P16, G17, N18, P20, N21, R24, D25, R27, D28, S31, R32, T35, Q38, M39, Q42,L43, D44, N45, L46, E50, S51, E54, G58, S66, Q70, E74, E75, P78, Q79, N82, Q83, D84, P85, D86, A89, H90, N92, S93, E96, N97, T100, R102, L103, R106, R107, H109, R110, P113, E122, Q123, N126, A127, N129, Q132, E133, D144, I145, N148, E151, T155, M156, R159 and N160, and at least one lysine residue selected from the group consisting of K34, K49, K57, K88, K99, K117, K119 and K130 has been removed, preferably by substitution to any other amino acid residue, in particular an arginine.

It will be understood that any of the amino acid changes, in particular substitutions, specified in this section can be combined with any of the amino acid changes, prefererably substitutions specified in the other sections herein disclosing specific amino acid modifications.

Conjugate of the Invention Having a Non-Lysine Residue as Attachment Group

Based on the present disclosure the skilled person will be aware that amino acid residues comprising other attachment groups may be introduced and/or removed from hIL-10 with the amino acid sequence shown in SEQ ID NO 2, using the same approach as that illustrated above with lysine residues. For instance, one or more amino acid residues comprising an acid group (glutamic acid and aspartic acid), asparagine, tyrosine and cystein may be introduced into positions which in hIL-10 are occupied by amino acid residues having surface exposed side chains (i.e. the positions mentioned above to be of interest for introduction of lysine residues), or removed (preferably by substitution with any other amino acid residue). Preferably, the substitutions are conservative substitutions that do not have the same attachment groups for the relevant non-polypeptide moiety (e.g. it should be avoided to substitute an amino acid residue with an attachment group for a given polypeptide moiety with a different amino acid residue comprising an attachment group for said non-polypeptide moiety). For instance, Asp is substituted with Asn, Glu with Gln, Tyr with Phe, and Cys with Ser and vice versa. Such modified polypeptides may further be in vivo glycosylated.

For instance, conjugates according to this aspect include a non-polypeptide moiety reactive with an aspartic acid or glutamic acid residue and a polypeptide i) that

comprises an amino acid sequence that differs from the amino acid sequence shown in SEQ ID NO 2 in the introduction of at least one aspartic acid residue in a position selected from the group consisting of S1, P2, G3, Q4, G5, T6, Q7, S8, E9, N10, S11, T13, H14, P16, G17, N18, P20, N21, R24, D25, R27, D28, S31, R32, T35, Q38, M39, Q42, L43, D44, N45, L6, E50, S51, E54, G58, S66, Q70, E74, E75, P78, Q79, N82, Q83, D84, P85, D86, A89, H90, N92, S93, E96, N97, T100, R102, L103, R106, R107, H109, R110, P113, E122, Q123, N126, A127, N129, Q132, E133, D144, L145, N148, E151, T155, M156, R159, and N160,

comprises an amino acid sequence that differs from the amino acid sequence shown in SEQ ID NO 2 in the introduction of at least one glutamic acid residue selected from the group consisting of introduction of at least one glutamic acid residue in a position selected from the group consisting of S1, P2, G3, Q4, G5, T6, Q7, S8, E9, N10, S11, T13, H14, P16, G17, N18, P20, N21, R24, D25, R27, D28, S31, R32, T35, Q38, M39, Q42, L43, D44, N45, L46, E50, S51, E54, G58, S66, Q70, E74, E75, P78, Q79, N82, Q83, D84, P85, D86, A89, H90, N92, S93, E96, N97, T100, R102, L103, R106, R107, H109, R110, P113, E122, Q123, N126, A127, N129, Q132, E133, D144, I145, N148, E151, T155, M156, R159, and N160,

comprises an amino acid sequence that differs from the amino acid sequence shown in SEQ ID NO 2 in the removal, preferably by substitution, of at least one of the amino acid residues selected from the group consisting of E9, D25, D28, D41, D44, E50, E54, D55, E67, E74, E75, E81, D84, D86, E96, E 115, E122, E133, E142, D144 and E151, preferably the E residue is substituted with an N residue and the E residue with a Q residue.

Conjugate of the Invention Wherein the Non-Polypeptide Moiety is a Carbohydrate Moiety

In a further aspect the invention relates to a conjugate comprising a glycosylated polypeptide exhibiting IL-10 activity, which comprises an amino acid sequence that differs from that shown in SEQ ID NO 2 in that at least one non-naturally occurring glycosylation site has been introduced into the amino acid sequence.

A suitable N-glycosylation site may be introduced by introducing, preferably by substitution, an asparagine residue in a position occupied by an amino acid residue having more than 25% of its side chain exposed at the surface of the polypeptide, which position does not have a proline residue located in position +1 and +3 therefrom. If the amino acid residue located in position +2 is a serine or threonine, no further amino acid substitution need to be made. However, if this position is occupied by a different amino acid residue a serine or threonine residue need to be introduced.

Preferably, the glycosylation site(s) is/are introduced by way of a substitution selected from the group consisting of P2N+Q4S, P2N+Q4T, G3N+G5S, G3N+G5T, Q4N+T6S, Q4N, G5N+Q7S, G5N+Q7T, T6N, T6N+S8T, Q7N+E9S, Q7N+E9T, S8N+N10S, S8N+N10T, E9N, E9N+S11T, S11N+T13S, S11N, H14N+P16S, H14N+P16T, P16N+N18S, P16N+N18T, P20S, P20T, P20N+M22S, P20N+M22T, L23S, L23T, R24N+L26S, R24N+L26T, D25N+R27S, D25N+R27T, R27N+A29S, R27N+A29T, D28N+F30S, D28N+F30T, S31N+V33S, S31N+V33T, R32N+K34S, R32N+K34T, K34N+F36S, K34N+F36T, T35N+F37S, T35N+F37T, Q38N+K40S, Q38N+K40T, M39N+D41S, M39N+D41T, K40N+Q42S, K40N+Q42T, Q42N+D44S, Q42N+D44T, L43N+N45S, L43N+N45T, D44N+L46S, D44N+L46T, L47S, L47T, L46N+L48S, L46N+L48T, K49N, K49N+S51T, E50N+L52S, E50N+L52T, S51N+L53S, S51N+L53T, E54N+F56S, E54N+F56T, K57N+Y59S, K57N+Y59T, G58N+L60S, G58N+L60T, S66N+M68S, S66N+M68T, Q70N+Y72S, Q70N+Y72T, E74N+V76S, E74N+V76T, P78N+A80S, P78N+A80T, Q79N+E81S, Q79N+E81T, Q83N+P85S, Q83N+P85T, P85N+187S, P85N+187T, D86N+K88S, D86N+K88T, K88N+H90S, K88N+H90T, A89N+V91S, A89N+V91T, H90N+N92S, H90N+N92T, L94S, L94T, S93N+G95S, S93N+G95T, E96N+L98S, E96N+L98T, K99S, K99T, K99N+L101S, K99N+L101T, T100SN+R102S, T100N+R102T, R102N+R104S, R102N+R104T, L103N+L105S, L103N+L105T, R107N+H109S, R107N+H109T, H109N+F111S, H109N+F111T, P113N+E115S, P113N+E115T, K117N+K119S, K117N+K119T, E122N+V124S, E122N+V124T, Q123N+K125S, Q123N+K125T, K125N+A127S, K125N+A127T, F128S, F128T, A127N+N129S, A127N+N129T, L131 S, L131T, K130N+Q132S, K130N+Q132T, Q132N+K134S, Q132N+K134T, E133N+G135S, E133N+G135T, K134N+I136S, K134N+I136T, D144N+F146S, D144N+F146T, I145N+I147S, I145N+I147T, I150S, I150T, E151N+Y153S, E151N+Y153T, T155N+K157S, T155N+K157T, M156N+I158S, M156N+I158T, K157N+R159S and K157N+R159T, more preferably from the group consisting of P2N+Q4S, P2N+Q4T, G3N+G5S, G3N+G5T, Q4N+T6S, Q4N, G5N+Q7S, G5N+Q7T, T6N, T6N+S8T, Q7N+E9S, Q7N+E9T, S8N+N10S, S8N+N10T, E9N, E9N+S11T, S11N+T13S, S11N, H14N+P16S, H14N+P16T, P16N+N18S, P16N+N18T, P20S, P20T, P20N+M22S, P20N+M22T, L23S, L23T, R24N+L26S, R24N+L26T, D25N+R27S, D25N+R27T, D28N+F30S, D28N+F30T, S31N+V33S, S31N+V33T, R32N+K34S, R32N+K34T, K34N+F36S, K34N+F36T, Q38N+K40S, Q38N+K40T, M39N+D41S, M39N+D41T, Q42N+D44S, Q42N+D44T, D44N+L46S, D44N+L46T, L47S, L47T, K49N, K49N+S51T, E50N+L52S, E50N+L52T, E54N+F56S, E54N+F56T, K57N+Y59S, K57N+Y59T, G58N+L60S, G58N+L60T, Q70N+Y72S, Q70N+Y72T, E74N+V76S, E74N+V76T, Q79N+E81S, Q79N+E81T, P85N+I87S, P85N+I87T, D86N+K88S, D86N+K88T, K88N+H90S, K88N+H90T, A89N+V91S, A89N+V91T, H90N+N92S, H90N+N92T, S93N+G95S, S93N+G95T, E96N+L98S, E96N+L98T, K99N+L101S, K99N+L101T, T100SN+R102S, T100N+R102T, L103N+L105S, L103N+L105T, R107N+H109S, R107N+H109T, H109N+F111S, H109N+F111T, K117N+K119S, K117N+K119T, E122N+V124S, E122N+V124T, F128S, F128T, A127N+N129S, A127N+N129T, L131S, L131T, K130N+Q132S, K130N+Q132T, Q132N+K134S, Q132N+K134T, E133N+G135S, E133N+G135T, D144N+F146S, D144N+F146T, I150S, I150T, T155N+K157S and T155N+K157T (each of these mutations being introducing a glycosylation site in a position occupied by an amino acid residue having more than 50% of its side chain surface exposed), even more preferably from the group consisting of Q4N, T6N, E9N, S 11N, P20S, P20T, L23S, L23T, 147S, 147T, K49N, L94S, L94T, K99S, K99T, F128S, F128T, L131S, L131T, I150S, and I150T (requiring only one mutation to introduce glycosylation site), and most preferably from the group consisting of Q4N, T6N, E9N, S11N, L23S, L23T, L47S, L47T, K49N, F128S, F128T, L131S, L131T, I150S, and I150T (requiring only one mutation to introduce glycosylation site).

Preferably, the polypeptide comprises 1, 2, 3, 4, 5, 6, 7, 8, 9 or 10 introduced glycosylation sites.

The glycosylated polypeptide according to this aspect comprises at least one sugar moiety, but may contain more, e.g. 1-10 sugar moieties (depending on the number of glycosylation sites and the extent to which they are used).

Alternatively or additionally, the polypeptide i) may have an amino acid sequence which differs from that of SEQ ID NO 2 in at least one removed, naturally occurring N-glycosylation site. For instance, the conjugate comprises a polypeptide comprising an amino acid sequence that differs from the amino acid sequence shown in SEQ ID NO 2 in the removal, preferably by substitution, of at least one of the amino acid residues selected from the group consisting of N116 and S118. N116 may be substituted with any other amino acid residue, and S118 with any other amino acid residue than T. Preferably, the N residue is substituted with Q or D, and the T residue with A or G.

Furthermore, the amino acid sequence of polypeptide i) having at least one of the above mentioned N-glycosylation site modifications may differ from that of SEQ ID NO 2 in the removal of at least one lysine residue as identified above in the section entitled “Removal of lysine residue(s)”.

It will be understood that in order to prepare a conjugate according to this aspect the polypeptide i) must be expressed in a glycosylating host cell capable of attaching sugar moieties at the glycosylation site(s) or alternatively subjected to in vitro glycosylation. Examples of glycosylating host cells are given in the section further below entitled “Coupling to a sugar moiety”.

In addition to a carbohydrate molecule, the conjugate according to the aspect of the invention described in the present section may contain additional non-polypeptide moieties, in particular a polymer molecule, as described in the present application, conjugated to one or more, optionally introduced attachment groups present in the polypeptide part of the conjugate.

It will be understood that any of the amino acid changes, in particular substitutions, specified in this section can be combined with any of the amino acid changes, in particular substitutions, specified in the other sections herein disclosing specific amino acid changes.

Non-Polypeptide Moiety of the Conjugate of the Invention

As indicated further above the non-polypeptide moiety of the conjugate of the invention is preferably selected from the group consisting of a polymer molecule, a lipophilic compound, a sugar moiety (e.g. by way of in vivo glycosylation) and an organic derivatizing agent. All of these agents may confer desirable properties to the polypeptide part of the conjugate, in particular an increased functional in vivo half-life and/or an increased serum half-life. The polypeptide part of the conjugate is normally conjugated to only one type of non-polypeptide moiety, but may also be conjugated to two or more different types of non-polypeptide moieties, e.g. to a polymer molecule and a sugar moiety, to a lipophilic group and a sugar moiety, to an organic derivatizing agent and a sugar moiety, to a lipophilic group and a polymer molecule, etc. The conjugation to two or more different non-polypeptide moieties may be done simultaneous or sequentially.

Polypeptide of the Invention

In a further aspect the invention relates to a polypeptide exhibiting IL-10 activity, which constitutes part of a conjugate of the invention, and is as described in the section entitled “Conjugate of the invention”. Said polypeptide is preferably glycosylated and thus further comprises N-linked and/or O-linked sugar moieties.

Methods of Preparing a Conjugate of the Invention

In the following sections “Conjugation to a lipophilic compound”, “Conjugation to a polymer molecule”, “Conjugation to a sugar moiety” and “Conjugation to an organic derivatizing agent” conjugation to specific types of non-polypeptide moieties is described.

Conjugation to a Lipophilic Compound

The polypeptide and the lipophilic compound may be conjugated to each other, either directly or by use of a linker. The lipophilic compound may be a natural compound such as a saturated or unsaturated fatty acid, a fatty acid diketone, a terpene, a prostaglandin, a vitamine, a carotenoide or steroide, or a synthetic compound such as a carbon acid, an alcohol, an amine and sulphonic acid with one or more alkyl-, aryl-, alkenyl- or other multiple unsaturated compounds. The conjugation between the polypeptide and the lipophilic compound, optionally through a linker may be done according to methods known in the art, e.g. as described by Bodanszky in Peptide Synthesis, John Wiley, New York, 1976 and in WO 96/12505.

Conjugation to a Polymer Molecule

The polymer molecule to be coupled to the polypeptide may be any suitable polymer molecule, such as a natural or synthetic homo-polymer or hetero-polymer, typically with a molecular weight in the range of 300-100,000 Da, such as 300-20,000 Da, more preferably in the range of 500-10,000 Da, even more preferably in the range of 500-5000 Da. Examples of homo-polymers include a polyol (i.e. poly-OH), a polyamine (i.e. poly-NH₂) and a polycarboxylic acid (i.e. poly-COOH). A hetero-polymer is a polymer which comprises different coupling groups, such as a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-co-maleic acid anhydride, polystyrene-co-maleic acid anhydride, dextran, including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG is the preferred polymer molecule, since it has only few reactive groups capable of cross-linking compared to e.g. polysaccharides such as dextran. In particular, monofunctional PEG, e.g. methoxypolyethylene glycol (m-PEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e. with reactive functional groups (examples of which include primary amino groups, hydrazide (HZ), thiol, succinate (SUC), succinimidyl succinate (SS), succinimidyl succinamide (SSA), succinimidyl proprionate (SPA), succinimidy carboxymethylate (SCM), benzotriazole carbonate (BTC), N-hydroxysuccinimide (NHS), aldehyde, nitrophenylcarbonate (NPC), and tresylate (TRES)). Suitable activated polymer molecules are commercially available, e.g. from Shearwater Polymers, Inc., Huntsville, Ala., USA. Alternatively, the polymer molecules can be activated by conventional methods known in the art, e.g. as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, AID-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575, both of which are incorporated herein by reference. Furthermore, the following publications, incorporated herein by reference, disclose useful polymer molecules and/or PEGylation chemistries: U.S. Pat. No. 5,824,778, U.S. Pat. No. 5,476,653, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Florida, USA; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. being amine, hydroxyl, carboxyl, aldehyde, sulfydryl, succinimidyl, maleimide, vinysulfone or haloacetate). The PEGylation may be directed towards conjugation to all available attachment groups on the polypeptide (i.e. such attachment groups that are exposed at the surface of the polypeptide) or may be directed towards one or more specific attachment groups, e.g. the N-terminal amino group (U.S. Pat. No. 5,985,265). Furthermore, the conjugation may be achieved in one step or in a stepwise manner (e.g. as described in WO 99/55377).

It will be understood that the PEGylation is designed so as to produce the optimal molecule with respect to the number of PEG molecules attached, the size and form of such molecules (e.g. whether they are linear or branched), and the attachment site(s) in the polypeptide. The molecular weight of the polymer to be used may e.g. be chosen on the basis of the desired effect to be achieved. For instance, if the primary purpose of the conjugation is to achieve a conjugate having a high molecular weight (e.g. to reduce renal clearance) it is usually desirable to conjugate as few high Mw polymer molecules as possible to obtain the desired molecular weight. When a high degree of epitope shielding is desirable this may be obtained by use of a sufficiently high number of low molecular weight polymer molecules (e.g. with a molecular weight of about 5000 Da) to effectively shield all or most epitopes of the polypeptide. For instance, 2-8, such as 3-6 such polymers may be used.

In connection with conjugation to only a single attachment group on the protein (as described in U.S. Pat. No. 5,985,265), it may be advantageous that the polymer molecule, which may be linear or branched, has a high molecular weight, e.g. about 20 kDa.

Normally, the polymer conjugation is performed under conditions aimed at reacting all available polymer attachment groups with polymer molecules, in particular by using a molar excess of the non-polypeptide moiety relative to the polypeptide. Typically, the molar ratio of activated polymer molecules to polypeptide is up to about 1000-1, in particular up to about 200-1, preferably up to about 100-1, such as up to about 10-1 or 5-1 in order to obtain optimal reaction.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378).

Subsequent to the conjugation, residual activated polymer molecules are blocked according to methods known in the art, e.g. by addition of primary amine to the reaction mixture, and the resulting inactivated polymer molecules are removed by a suitable method.

In a specific embodiment, the polypeptide conjugate of the invention is one which comprises a single PEG molecule attached to the N-terminal of the polypeptide and no other PEG molecules, in particular a linear or branched PEG molecule with a molecular weight of at least about 20 kDa. The polypeptide according to this embodiment may further comprise one or more oligosaccharide moieties attached to an N-linked or O-linked glycosylation site of the polypeptide or carbohydrate moieties attached by in vitro glycosylation.

In another specific embodiment, the polypeptide conjugate of the invention comprises a PEG molecule attached to each of the lysine residues in the polypeptide available for PEGylation, in particular a linear or branched PEG molecule, e.g. with a molecular weight of about 5 kDa.

In yet another embodiment, the polypeptide conjugate of the invention comprises a PEG molecule attached to each of the lysine residues in the polypeptide available for PEGylation, and in addition to the N-terminal amino acid residue of the polypeptide.

Covalent in vitro coupling of carbohydrate moieties (such as dextran) to amino acid residues of the polypeptide may also be used, e.g. as described, for example in WO 87/05330 and in Aplin etl al., CRC Crit Rev. Biochem, pp. 259-306, 1981. The in vitro coupling of carbohydrate moieties or PEG to protein- and peptide-bound Gln-residues can be carried out by transglutaminases (TGases). Transglutaminases catalyse the transfer of donor amine-groups to protein- and peptide-bound Gln-residues in a so-called cross-linking reaction. The donor-amine groups can be protein- or peptide-bound e.g. as the ε-amino-group in Lys-residues or it can be part of a small or large organic molecule. An example of a small organic molecule functioning as amino-donor in TGase-catalysed cross-linking is putrescine (1,4-diaminobutane). An example of a larger organic molecule functioning as amino-donor in TGase-catalysed cross-linking is an amine-containing PEG (Sato et al., 1996, Biochemistry 35, 13072-13080).

TGases, in general, are highly specific enzymes, and not every Gln-residues exposed on the surface of a protein is accessible to TGase-catalysed cross-linking to amino-containing substances. On the contrary only few Gln-residues are naturally functioning as TGase substrates but the exact parameters governing which Gln-residues are good TGase substrates remain unknown. Thus, in order to render a protein susceptible to TGase-catalysed cross-linking reactions it is often a prerequisite at convenient positions to add stretches of amino acid sequence known to function very well as TGase substrates. Several amino acid sequences are known to be or to contain excellent natural TGase substrates e.g. substance P, elafin, fibrinogen, fibronectin, α₂-plasmin inhibitor, α-caseins, and β-caseins.

Coupling to a Sugar Moiety

The conjugation to a sugar moiety takes place by in vivo glycosylation effected by a glycosylating, eucaryotic expression host. The expression host cell may be selected from fungal (filamentous fungal or yeast), insect or animal cells or from transgenic plant cells. In one embodiment the host cell is a mammalian cell, such as a CHO cell, BHK or HEK, e.g. HEK 293, cell, or an insect cell, such as an SF9 cell, or a yeast cell, e.g. S. cerevisiae or Pichia pastoris, or any of the host cells mentioned hereinafter.

Coupling to an Organic Derivatizing Agent

Covalent modification of the polypeptide exhibiting IL-10 activity may be performed by reacting (an) attachment group(s) of the polypeptide with an organic derivatizing agent. Suitable derivatizing agents and methods are well known in the art. For example, cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(4-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri4-nitrophenol, or chloro-7-nitrobenzo-2-oxa-1,3-diazole. Histidyl residues are derivatized by reaction with diethylpyrocarbonateat pH 5.5-7.0 because this agent is relatively specific for the histidyl side chain. Para-bromophenacyl bromide also is useful. The reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group.

Furthermore, these reagents may react with the groups of lysine as well as the arginine guanidino group. Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Blocking of Functional Site

It has been reported that excessive polymer conjugation can lead to a loss of activity of the polypeptide to which the polymer is conjugated. This problem can be eliminated, e.g., by removal of attachment groups located at the functional site or by blocking the functional site prior to conjugation so that the functional site is blocked during conjugation. The latter strategy constitutes further embodiments of the invention (the first strategy being exemplified further above, e.g. by removal of lysine residues which may be located close to the functional site). More specifically, according to the second strategy the conjugation between the polypeptide and the non-polypeptide moiety ii) is conducted under conditions where the functional site of the polypeptide i) is blocked by a helper molecule capable of binding to the functional site of the polypeptide i).

Preferably, the helper molecule is one, which specifically recognizes a functional site of the polypeptide, such as a receptor, in particular the IL-10 receptor or a part of the 1L-10 receptor.

Alternatively, the helper molecule may be an antibody, in particular a monoclonal antibody recognizing the polypeptide exhibiting IL-10 activity. In particular, the helper molecule may be a neutralizing monoclonal antibody.

The polypeptide is allowed to interact with the helper molecule before effecting conjugation. This ensures that the functional site of the polypeptide is shielded or protected and consequently unavailable for derivatization by the non-polypeptide moiety such, as a polymer. Following its elution from the helper molecule, the conjugate between the non-polypeptide moiety and the polypeptide can be recovered with at least a partially preserved functional site.

The subsequent conjugation of the polypeptide having a blocked functional site to a polymer, a lipophilic compound, a sugar moiety, an organic derivatizing agent or any other compound is conducted in the normal way, e.g. as described in the sections above entitled “Conjugation to . . . ”.

Irrespectively of the nature of the helper molecule to be used to shield the functional site of the polypeptide from conjugation, it is desirable that the helper molecule is free from or comprises only a few attachment groups for the non-polypeptide moiety of choice in part(s) of the molecule, where the conjugation to such groups will hamper the desorption of the conjugated polypeptide from the helper molecule. Hereby, selective conjugation to attachment groups present in non-shielded parts of the polypeptide can be obtained and it is possible to reuse the helper molecule for repeated cycles of conjugation. For instance, if the non-polypeptide moiety is a polymer molecule such as PEG, which has the epsilon amino group of a lysine or N-terminal amino acid residue as an attachment group, it is desirable that the helper molecule is substantially free from conjugatable epsilon amino groups, preferably free from any epsilon amino groups. Accordingly, in a preferred embodiment the helper molecule is a protein or peptide capable of binding to the functional site of the polypeptide, which protein or peptide is free from any conjugatable attachment groups for the non-polypeptide moiety of choice.

In a further embodiment the helper molecule is first covalently linked to a solid phase such as column packing materials, for instance Sephadex or agarose beads, or a surface, e.g. reaction vessel. Subsequently, the polypeptide is loaded onto the column material carrying the helper molecule and conjugation carried out according to methods known in the art, e.g. as described in the sections above entitled “Conjugation to . . . ”. This procedure allows the polypeptide conjugate to be separated from the helper molecule by elution. The polypeptide conjugate is eluated by conventional techniques under physico-chemical conditions that do not lead to a substantive degradation of the polypeptide conjugate. The fluid phase containing the polypeptide conjugate is separated from the solid phase to which the helper molecule remains covalently linked. The separation can be achieved in other ways: For instance, the helper molecule may be derivatised with a second molecule (e.g. biotin) that can be recognized by a specific binder (e.g. streptavidin). The specific binder may be linked to a solid phase thereby allowing the separation of the polypeptide conjugate from the helper molecule-second molecule complex through passage over a second helper-solid phase column which will retain, upon subsequent elution, the helper molecule-second molecule complex, but not the polypeptide conjugate. The polypeptide conjugate may be released from the helper molecule in any appropriate fashion. Deprotection may be achieved by providing conditions in which the helper molecule dissociates from the functional site of the IL-10 to which it is bound. For instance, a complex between an antibody to which a polymer is conjugated and an anti-idiotypic antibody can be dissociated by adjusting the pH to an acid or alkaline pH.

Conjugation of a Tagged Polypeptide i)

In an alternative embodiment the polypeptide i) is expressed, as a fusion protein, with a tag, i.e. an amino acid sequence or peptide stretch made up of typically 1-30, such as 1-20 amino acid residues. Besides allowing for fast and easy purification, the tag is a convenient tool for achieving conjugation between the tagged polypeptide i) and the non-polypeptide moiety ii). In particular, the tag may be used for achieving conjugation in microtiter plates or other carriers, such as paramagnetic beads, to which the tagged polypeptide can be immobilised via the tag. The conjugation to the tagged polypeptide i) in, e.g., microtiter plates has the advantage that the tagged polypeptide can be immobilised in the microtiter plates directly from the culture broth (in principle without any purification) and subjected to conjugation. Thereby, the total number of process steps (from expression to conjugation) can be reduced. Furthermore, the tag may function as a spacer molecule ensuring an improved accessibility to the immobilised polypeptide to be conjugated. The conjugation using a tagged polypeptide i) may be to any of the non-polypeptide moieties disclosed herein, e.g. to a polymer molecule such as PEG.

The identity of the specific tag to be used is not critical as long as the tag is capable of being expressed with the polypeptide i) and is capable of being immobilised on a suitable surface or carrier material. A number of suitable tags are commercially available, e.g. from Unizyme Laboratories, Denmark. For instance, the tag may consist of any of the following sequences:

His-His-His-His-His-His
Met-Lys-His-His-His-His-His-His
Met-Lys-His-His-Ala-His-His-Gln-His-His
Met-Lys-His-Gln-His-Gln-His-Gln-His-Gln-His-Gln-
His-Gln
Met-Lys-His-Gln-His-Gln-His-GIn-His-Gln-His-Gln-
His-Gln-Gln

or any of the following:

EQKLISEEDL (a C-terminal tag described in Mol. Cell. Biol. 5:3610-16, 1985)

DYKDDDDK (a C- or N-terminal tag)

YPYDVPDYA

Antibodies against the above tags are commercially available, e.g. from ADI, Aves Lab and Research Diagnostics.

A convenient method for using a tagged polypeptide for PEGylation is given in the Methods section below. The subsequent cleavage of the tag from the polypeptide i) may be achieved by use of commercially available enzymes.

Methods of Preparing a Polypeptide of the Invention or the Polypeptide i) of the Conjugate of the Invention

The polypeptide of the present invention or the polypeptide part of a conjugate of the invention, optionally in glycosylated form, may be produced by any suitable method known in the art. Such methods include constructing a nucleotide sequence encoding the polypeptide and expressing the sequence in a suitable transformed or transfected host. However, polypeptides of the invention may be produced, albeit less efficiently, by chemical synthesis or a combination of chemical synthesis or a combination of chemical synthesis and recombinant DNA technology.

The nucleotide sequence encoding a polypeptide with the SEQ ID NO 1 or 2, or the polypeptide i) of a conjugate of the invention may be constructed by isolating or synthesizing a nucleotide sequence encoding the parent IL-10, such as hIL-10 with the amino acid sequence shown in SEQ ID NO 1 or 2 and then changing the nucleotide sequence so as to effect introduction (i.e. insertion or substitution) or deletion (i.e. removal or substitution) of the relevant amino acid residue(s). Such nucleotide sequences are available in the art or can be synthesized by the skilled person. The nucleotide sequence is conveniently modified by site-directed mutagenesis in accordance with conventional methods. Alternatively, the nucleotide sequence is prepared by chemical synthesis, e.g. by using an oligonucleotide synthesizer, wherein oligonucleotides are designed based on the amino acid sequence of the desired polypeptide, and preferably selecting those codons that are favored in the host cell in which the recombinant polypeptide will be produced. For example, several small oligonucleotides coding for portions of the desired polypeptide may be synthesized and assembled by PCR, ligation or ligation chain reaction (LCR) (Barany, PNAS 88:189-193, 1991). The individual oligonucleotides typically contain 5′ or 3′ overhangs for complementary assembly.

Once assembled (by synthesis, site-directed mutagenesis or another method), the nucleotide sequence encoding the polypeptide is inserted into a recombinant vector and operably linked to control sequences necessary for expression of the IL-10 in the desired transformed host cell.

It should of course be understood that not all vectors and expression control sequences function equally well to express the nucleotide sequence encoding a polypeptide described herein. Neither will all hosts function equally well with the same expression system. However, one of skill in the art may make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector, the toxicity of the product coded for by the nucleotide sequence, their secretion characteristics, their ability to fold the polypeptide correctly, their fermentation or culture requirements, and the ease of purification of the products coded for by the nucleotide sequence.

The recombinant vector may be an autonomously replicating vector, i.e. a vector, which exists as an extrachromosomal entity, the replication of which is independent of chromosomal replication, e.g. a plasmid. Alternatively, the vector is one which, when introduced into a host cell, is integrated into the host cell genome and replicated together with the chromosome(s) into which it has been integrated.

The vector is preferably an expression vector, in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for eukaryotic hosts, include, for example, vectors comprising expression control sequences from SV40, bovine papilloma virus, adenovirus and cytomegalovirus. Specific vectors are, e.g., pCDNA3.1(+)\Hyg (Invitrogen, Carlsbad, Calif., USA) and pCI-neo (Stratagene, La Jola, Calif., USA). Useful expression vectors for yeast cells include the 2μplasmid and derivatives thereof, the POT1 vector (U.S. Pat. No. 4,931,373), the pJSO37 vector described in Okkels, Ann. New York Acad. Sci. 782, 202-207, 1996, and pPICZ A, B or C (Invitrogen). Useful vectors for insect cells include pVL941, pBG311 (Cate et al., “Isolation of the Bovine and Human Genes for Mullerian Inhibiting Substance And Expression of the Human Gene In Animal Cells”, Cell, 45, pp. 685-98 (1986), pBluebac 4.5 and pMe1bac (both available from Invitrogen). Useful expression vectors for bacterial hosts include known bacterial plasmids, such as plasmids from E. coli, including pBR322, pET3a and pET12a (both from Novagen Inc., WI, USA), wider host range plasmids, such as RP4, phage DNAs, e.g., the numerous derivatives of phage lambda, e.g., NM989, and other DNA phages, such as M13 and filamentous single stranded DNA phages.

Other vectors for use in this invention include those that allow the nucleotide sequence encoding the polypeptide to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrafolate Reductase cDNA Gene: Analysis Of Signals Utilized For Efficient Expression”, Mol. Cell. Biol., 2, pp. 1304-19 (1982)) and glutamine synthetase (“GS”) amplification (see, e.g., U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For Saccharomyces cerevisiae, selectable markers include ura3 and leu2. For filamentous fungi, selectable markers include amdS, pyrG, arcB, niaD and sC.

The term “control sequences” is defined herein to include all components, which are necessary or advantageous for the expression of the polypeptide of the invention. Each control sequence may be native or foreign to the nucleic acid sequence encoding the polypeptide. Such control sequences include, but are not limited to, a leader sequence, polyadenylation sequence, propeptide sequence, promoter, enhancer or upstream activating sequence, signal peptide sequence, and transcription terminator. At a minimum, the control sequences include a promoter.

A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g. the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus Elb region polyadenylation signals and the Kozak consensus sequence (Kozak, M. J Mol Biol Aug. 20, 1987;196(4):947-50).

In order to improve expression in mammalian cells a synthetic intron may be inserted in the 5′ untranslated region of the nucleotide sequence encoding the polypeptide. An example of a synthetic intron is the synthetic intron from the plasmid pCI-Neo (available from Promega Corporation, WI, USA).

Examples of suitable control sequences for directing transcription in insect cells include the polyhedrin promoter, the P10 promoter, the Autographa californica polyhedrosis virus basic protein promoter, the baculovirus immediate early gene 1 promoter and the baculovirus 39K delayed-early gene promoter, and the SV40 polyadenylation sequence. Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4c promoter, and the inducible GAL promoter. Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator. Examples of suitable control sequences for use in bacterial host cells include promoters of the lac system, the trp system, the TAC or TRC system, and the major promoter regions of phage lambda.

The presence or absence of a signal peptide will, e.g., depend on the expression host cell used for the production of the polypeptide to be expressed (whether it is an intracellular or extracellular polypeptide) and whether it is desirable to obtain secretion. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide may conveniently be derived from an insect gene (cf. WO 90/05783), such as the Lepidopteran manduca sexta adipokinetic hormone precursor, (cf. U.S. Pat. No. 5,023,328), the honeybee melittin (Invitrogen), ecdysteroid UDP glucosyltransferase (egt) (Murphy et al., Protein Expression and Purification 4, 349-357 (1993) or human pancreatic lipase (hpl) (Methods in Enzymology 284, pp. 262-272, 1997). A preferred signal peptide for use in mammalian cells is that of hIL-10 or the murine Ig kappa light chain signal peptide (Coloma, M (1992) J. Imm. Methods 152:89-104). For use in yeast cells suitable signal peptides have been found to be the α-factor signal peptide from S. cereviciae (cf. U.S. Pat. No. 4,870,008), a modified carboxypeptidase signal peptide (cf. L. A. Valls et al., Cell 48, 1987, pp. 887-897), the yeast BAR1 signal peptide (cf. WO 87/02670), the yeast aspartic protease 3 (YAP3) signal peptide (cf. M. Egel-Mitani et al., Yeast 6, 1990, pp. 127-137), and the synthetic leader sequence TA57 (WO98/32867). For use in E. coli cells a suitable signal peptide have been found to be the signal peptide ompA (EP581821).

The nucleotide sequence of the invention encoding a polypeptide exhibiting IL-10 activity, whether prepared by site-directed mutagenesis, synthesis, PCR or other methods, may or may not also include a nucleotide sequence that encode a signal peptide. The signal peptide is present when the polypeptide is to be secreted from the cells in which it is expressed. Such signal peptide, if present, should be one recognized by the cell chosen for expression of the polypeptide. The signal peptide may be homologous (e.g. be that normally associated with hIL-10) or heterologous (i.e. originating from another source than hIL-10) to the polypeptide or may be homologous or heterologous to the host cell, i.e. be a signal peptide normally expressed from the host cell or one which is not normally expressed from the host cell. Accordingly, the signal peptide may be prokaryotic, e.g. derived from a bacterium such as E. coli, or eukaryotic, e.g. derived from a mammalian, or insect or yeast cell.

Any suitable host may be used to produce the polypeptide or polypeptide part of the conjugate of the invention, including bacteria, fungi (including yeasts), plant, insect, mammal, or other appropriate animal cells or cell lines, as well as transgenic animals or plants. Examples of bacterial host cells include grampositive bacteria such as strains of Bacillus, e.g. B. brevis or B. subtilis, Pseudomonas or Streptomyces, or gramnegative bacteria, such as strains of E. coli. The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278). Examples of suitable filamentous fungal host cells include strains of Aspergillus, e.g. A. oryzae, A. niger, or A. nidulans, Fusarium or Trichoderma. Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Examples of suitable yeast host cells include strains of Saccharomyces, e.g. S. cerevisiae, Schzizosaccharomyces, Klyveromyces, Pichia, such as P. pastoris or P. methanolica, Hansenula, such as H. Polymorpha or Yarrowia. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; Hinnen et al., 1978, Proceedings of the National Academy of Sciences USA 75: 1920: and as disclosed by Clontech Laboratories, Inc, Palo Alto, Calif., USA (in the product protocol for the Yeastmaker™ Yeast Transformation System Kit). Examples of suitable insect host cells include a Lepidoptora cell line, such as Spodoptera frugiperda (Sf9 or Sf21) or Trichoplusioa ni cells (High Five) (U.S. Pat. No. 5,077,214). Transformation of insect cells and production of heterologous polypeptides therein may be performed as described by Invitrogen. Examples of suitable mammalian host cells include Chinese hamster ovary (CHO) cell lines, (e.g. CHO-K1; ATCC CCL-61), Green Monkey cell lines (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture. Additional suitable cell lines are known in the art and available from public depositories such as the American Type Culture Collection, Rockville, Md.

Also, the mammalian cell, such as a CHO cell, may be modified to express sialyltransferase, e.g. 1,6-sialyltransferase, e.g. as described in U.S. Pat. No. 5,047,335, in order to provide improved glycosylation of the polypeptide.

Methods for introducing exogeneous DNA into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamin 2000. These methods are well known in the art and e.g. described by Ausbel et al. (eds.), 1996, Current Protocols in Molecular Biology, John Wiley & Sons, New York, USA. The cultivation of mammalian cells are conducted according to established methods, e.g. as disclosed in (Animal Cell Biotechnology, Methods and Protocols, Edited by Nigel Jenkins, 1999, Human Press Inc, Totowa, N.J., USA and Harrison Mass. and Rae IF, General Techniques of Cell Culture, Cambridge University Press 1997).

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermenters performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The resulting polypeptide may be recovered by methods known in the art. For example, the polypeptide may be recovered from the nutrient medium by conventional procedures including, but not limited to, centrifugation, filtration, extraction, spray drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989). Specific methods for purifying polypeptides exhibiting IL-10 activity are described (Human Cytokines, Handbook of Basic and Clinical Research, Volume II, Blackwell Science, Eds. Aggarwal and Gutterman, 1996, pp. 19-42).

Homogeneous Preparation of a Conjugate of the Invention

In a further aspect the invention relates to a substantially homogeneous preparation of a conjugate of the invention. In the present context a “substantially homogeneous preparation” is a preparation containing more than 50%, such as more than 75% and preferably more than 85%, or more than 90% identical conjugates, i.e. having the same degree and nature of conjugation. The substantially homogeneous preparation is conveniently obtained by ensuring that the polypeptide part of the conjugate contains the necessary number of attachment groups, located at the surface of the molecule in such a way that all attachment groups can be conjugated to the non-polypeptide moiety of choice, when the conjugation is performed in the presence of a molar excess of the non-polypeptide moiety relative to the polypeptide. Preferably, the non-polypeptide moiety to be used in this aspect of the invention is a polymer molecule.

Pharmaceutical Composition of the Invention and Its Use

Therapeutic formulations of the polypeptide or conjugate of the invention are typically administered in a composition that includes one or more pharmaceutically acceptable carriers or excipients. Such pharmaceutical compositions may be prepared in a manner known per se in the art to result in a polypeptide pharmaceutical that is sufficiently storage-stable and is suitable for administration to humans or animals.

The polypeptide or conjugate of the invention can be used “as is” and/or in a salt form thereof. Suitable salts include, but are not limited to, salts with alkali metals or alkaline earth metals, such as sodium, potassium, calcium and magnesium, as well as e.g. zinc salts. These salts or complexes may by present as a crystalline and/or amorphous structure.

“Pharmaceutically acceptable” means a carrier or excipient that at the dosages and concentrations employed does not cause any untoward effects in the patients to whom it is administered. Such pharmaceutically acceptable carriers and excipients are well known in the art (see Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]).

The polypeptides and conjugates of the invention will be administered to patients in a therapeutically effective dose. By “therapeutically effective dose” herein is meant a dose that is sufficient to produced the desired effects in relation to the condition for which it is administered. The exact dose will depend on the disorder to be treated, and will be ascertainable by one skilled in the art using known techniques. Normally, the dose approximately parallelels that employed in therapy with rhIL-10 such as Tenovil®, or a higher dosis. The exact dose to be administered depends on the circumstances. Normally, the dose should be capable of preventing or lessening the severity or spread of the condition or indication being treated. It will be apparent to those of skill in the art that an effective amount of a polypeptide, conjugate or composition of the invention depends, inter alia, upon the disease, the dose, the administration schedule, whether the polypeptide or conjugate or composition is administered alone or in conjunction with other therapeutic agents, the serum half-life of the compositions, and the general health of the patient. Preferably, the polypeptide, conjugate, preparation or composition of the invention is administered in an effective dose, in particular a dose which is sufficient to normalize the number of leukocytes, in particular neutrophils, in the patient in question. Normalization of the number of leukocytes may be determined by simply counting the number of leukocytes at regular intervals in accordance with established practice.

The pharmaceutical composition of the invention may be administered alone or in conjunction with other therapeutic agents. These agents may be incorporated as part of the same pharmaceutical composition or may be administered separately from the polypeptide or conjugate of the invention, either concurrently or in accordance with another treatment schedule. In addition, the polypeptide, conjugate or pharmaceutical composition of the invention may be used as an adjuvant to other therapies.

A “patient” for the purposes of the present invention includes both humans and other mammals. Thus the methods are applicable to both human therapy and veterinary applications.

The pharmaceutical composition of the polypeptide or conjugate of the invention may be formulated in a variety of forms, e.g. as a liquid, gel, lyophilized, or as a compressed solid. The preferred form will depend upon the particular indication being treated and will be apparent to one skilled in the art.

The administration of the formulations of the present invention can be performed in a variety of ways, including, but not limited to, orally, subcutaneously, intravenously, intranasally, transdermally, intraperitoneally, intramuscularly, intrapulmonary, or in any other acceptable manner. The formulations can be administered continuously by infusion, although bolus injection is acceptable, using techniques well known in the art, such as pumps or implantation. In some instances the formulations may be directly applied as a solution or spray.

Parenterals

An example of a pharmaceutical composition is a solution designed for parenteral administration. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are prepared for storage as lyophilized formulations or aqueous solutions by mixing, as appropriate, the polypeptide having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives are added to retard microbial growth, and are typically added in amounts of about 0.2%-1% (w/v). Suitable preservatives for use with the present invention include phenol, benzyl alcohol, meta-cresol, methyl paraben, propyl paraben, octadecyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol.

Isotonicifiers are added to ensure isotonicity of liquid compositions and include polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, omithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® polyols, polyoxyethylene sorbitan monoethers (Tween®-20, Tween®-80, etc.).

Additional miscellaneous excipients include bulking agents or fillers (e.g. starch), chelating agents (e.g. EDTA), antioxidants (e.g., ascorbic acid, methionine, vitamin E) and cosolvents. The active ingredient may also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences, supra.

Parenteral formulations to be used for in vivo administration must be sterile. This is readily accomplished, for example, by filtration through sterile filtration membranes.

Sustained Release Preparations

Suitable examples of sustained-release preparations include semi-permeable matrices of solid hydrophobic polymers containing the polypeptide or conjugate, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the ProLease® technology or Lupron Depot® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for long periods such as up to or over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S-S bond formation through thio-disulfide interchange, stabilization may be achieved by modifying sulfhydryl residues, lyophilizing from acidic solutions, controlling moisture content, using appropriate additives, and developing specific polymer matrix compositions.

Oral Administration

For oral administration, the pharmaceutical composition may be in solid or liquid form, e.g. in the form of a capsule, tablet, suspension, emulsion or solution. The pharmaceutical composition is preferably made in the form of a dosage unit containing a given amount of the active ingredient. A suitable daily dose for a human or other mammal may vary widely depending on the condition of the patient and other factors, but can be determined by persons skilled in the art using routine methods.

Solid dosage forms for oral administration may include capsules, tablets, suppositories, powders and granules. In such solid dosage forms, the active compound may be admixed with at least one inert diluent such as sucrose, lactose, or starch. Such dosage forms may also comprise, as is normal practice, additional substances, e.g. lubricating agents such as magnesium stearate. In the case of capsules, tablets and pills, the dosage forms may also comprise buffering agents. Tablets and pills can additionally be prepared with enteric coatings.

The polypeptides or conjugates may be admixed with adjuvants such as lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, they may be dissolved in saline, water, polyethylene glycol, propylene glycol, ethanol, oils (such as corn oil, peanut oil, cottonseed oil or sesame oil), tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be subjected to conventional pharmaceutical operations such as sterilization and/or may contain conventional adjuvants such as preservatives, stabilizers, wetting agents, emulsifiers, buffers, fillers, etc., e.g. as disclosed elsewhere herein. Liquid dosage forms for oral administration may include pharmaceutically acceptable emulsions, solutions, suspensions, syrups and elixirs containing inert diluents commonly used in the art, such as water. Such compositions may also comprise adjuvants such as wetting agents, sweeteners, flavoring agents and perfuming agents.

Suppositories

Suppositories for rectal administration of the polypeptide or conjugate can be prepared by mixing the active compound with a suitable non-irritating excipient such as cocoa butter and polyethylene glycols that are solid at ordinary temperatures but liquid at the rectal temperature and will therefore melt in the rectum and release the drug.

Topical Administration

Formulations suitable for topical administration include liquid or semi-liquid preparations suitable for penetration through the skin (e.g., liniments, lotions, ointments, creams, or pastes) and drops suitable for administration to the eye, ear, or nose.

Pulmonary Delivery

Formulations suitable for use with a nebulizer, either jet or ultrasonic, will typically comprise the polypeptide or conjugate dissolved in water at a concentration of, e.g., about 0.01 to 25 mg of conjugate per mL of solution, preferably about 0.1 to 10 mg/mL. The formulation may also include a buffer and a simple sugar (e.g., for protein stabilization and regulation of osmotic pressure), and/or human serum albumin ranging in concentration from 0.1 to 10 mg/ml. Examples of buffers that may be used are sodium acetate, citrate and glycine. Preferably, the buffer will have a composition and molarity suitable to adjust the solution to a pH in the range of 3 to 9. Generally, buffer molarities of from 1 mM to 50 mM are suitable for this purpose. Examples of sugars which can be utilized are lactose, maltose, mannitol, sorbitol, trehalose, and xylose, usually in amounts ranging from 1% to 10% by weight of the formulation.

The nebulizer formulation may also contain a surfactant to reduce or prevent surface induced aggregation of the protein caused by atomization of the solution in forming the aerosol. Various conventional surfactants can be employed, such as polyoxyethylene fatty acid esters and alcohols, and polyoxyethylene sorbitan fatty acid esters. Amounts will generally range between 0.001% and 4% by weight of the formulation. An especially preferred surfactant for purposes of this invention is polyoxyethylene sorbitan monooleate.

Specific formulations and methods of generating suitable dispersions of liquid particles of the invention are described in WO 9420069, U.S. Pat. No. 5,915,378, U.S. Pat. No. 5,960,792, U.S. Pat. No.

5,957,124, U.S. Pat. No. 5,934,272, U.S. Pat. No. 5,915,378, U.S. Pat. No. 5,855,564, U.S. Pat. No. 5,826,570 and U.S. Pat. No. 5,522,385 which are hereby incorporated by reference.

Formulations for use with a metered dose inhaler device will generally comprise a finely divided powder. This powder may be produced by lyophilizing and then milling a liquid conjugate formulation and may also contain a stabilizer such as human serum albumin (HSA). Typically, more than 0.5% (w/w) HSA is added. Additionally, one or more sugars or sugar alcohols may be added to the preparation if necessary. Examples include lactose maltose, mannitol, sorbitol, sorbitose, trehalose, xylitol, and xylose. The amount added to the formulation can range from about 0.01 to 200% (w/w), preferably from approximately 1 to 50%, of the conjugate present. Such formulations are then lyophilized and milled to the desired particle size.

The properly sized particles are then suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant. This mixture is then loaded into the delivery device. An example of a commercially available metered dose inhaler suitable for use in the present invention is the Ventolin metered dose inhaler, manufactured by Glaxo Inc., Research Triangle Park, N.C.

Formulations for powder inhalers will comprise a finely divided dry powder containing conjugate and may also include a bulking agent, such as lactose, sorbitol, sucrose, or mannitol in amounts which facilitate dispersal of the powder from the device, e.g., 50% to 90% by weight of the formulation. The particles of the powder shall have aerodynamic properties in the lung corresponding to particles with a density of about 1 g/cm² having a median diameter less than 10 micrometers, preferably between 0.5 and 5 micrometers, most preferably of between 1.5 and 3.5 micrometers. An example of a powder inhaler suitable for use in accordance with the teachings herein is the Spinhaler powder inhaler, manufactured by Fisons Corp., Bedford, Mass.

The powders for these devices may be generated and/or delivered by methods disclosed in U.S. Pat. No. 5,997,848, U.S. Pat. No. 5,993,783, U.S. Pat. No. 5,985,248, U.S. Pat. No. 5,976,574, U.S. Pat. No. 5,922,354, U.S. Pat. No. 5,785,049 and U.S. Pat. No. 55,654,007.

Mechanical devices designed for pulmonary delivery of therapeutic products, include but are not limited to nebulizers, metered dose inhalers, and powder inhalers, all of which are familiar to those of skill in the art. Specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; the Spinhaler powder inhaler, manufactured by Fisons Corp., Bedford, Mass.; the “standing cloud” device of Inhale Therapeutic Systems, Inc., San Carlos, Calif.; the AIR inhaler manufactured by Alkermes, Cambridge, Mass.; and the AERx pulmonary drug delivery system manufactured by Aradigm Corporation, Hayward, Calif.

In one aspect the polypeptide, the conjugate or the pharmaceutical composition according to the invention is used for the manufacture of a medicament for treatment of diseases, in particular inflammatory diseases, such as rheumatoid arthritis or Crohn's disease, and in connection with transplantation, immunodeficiencies and parasitic infections.

In another aspect the polypeptide, the conjugate or the pharmaceutical composition according to the invention is used in a method of treating a mammal having an inflammatory disease, such as rheumatoid arthritis, and in connection with transplantation, immunodeficiencies and parasitic infections, comprising administering to a mammal in need thereof such a polypeptide, conjugate or pharmaceutical composition.

Materials and Methods

Methods Used to Determine the Amino Acids to be Modified

Accessible Surface Area (ASA)

The computer program Access (B. Lee and F. M. Richards, J. Mol. Biol. 55: 379400 (1971)) version 2 (Copyright (c) 1983 Yale University) are7 used to compute the accessible surface area (ASA) of the individual atoms in the structure. This method typically uses a probe-size of 1.4 Å and defines the Accessible Surface Area (ASA) as the area formed by the centre of the probe. Prior to this calculation all water molecules and all hydrogen atoms should be removed from the coordinate set, as should other atoms not directly related to the protein.

Fractional ASA of Side Chain

The fractional ASA of the side chain atoms is computed by division of the sum of the ASA of the atoms in the side chain with a value representing the ASA of the side chain atoms of that residue type in an extended ALA-x-ALA tripeptide. See Hubbard, Campbell & Thornton (1991) J. Mol. Biol. 220,507-530. For this example the CA atom is regarded as a part of the side chain of Glycine residues but not for the remaining residues. The following table are used as standard 100% ASA for the side chain:

| | | |
|---|---|---|
| Ala | 69.23 | Å² |
| Arg | 200.35 | Å² |
| Asn | 106.25 | Å² |
| Asp | 102.06 | Å² |
| Cys | 96.69 | Å² |
| Gln | 140.58 | Å² |
| Glu | 134.61 | Å² |
| Gly | 32.28 | Å² |
| His | 147.00 | Å² |
| Ile | 137.91 | Å² |
| Leu | 140.76 | Å² |
| Lys | 162.50 | Å² |
| Met | 156.08 | Å² |
| Phe | 163.90 | Å² |
| Pro | 119.65 | Å² |
| Ser | 78.16 | Å² |
| Thr | 101.67 | Å² |
| Trp | 210.89 | Å² |
| Tyr | 176.61 | Å² |
| Val | 114.14 | Å² |

Residues not detected in the structure are defined as having 100% exposure as they are thought to reside in flexible regions.

Determining Distances Between Atoms

The distance between atoms is most easily determined using molecular graphics software e.g. InsightII v. 98.0, MSI INC.

Determining Surface Exposed Atoms

The X-ray structure of human IL10 (Zdanov A., Schalk-Hihi C., Wlodawer A.,: “Crystal structure of human interleukin-10 at 1.6 Å resolution and a model of a complex with its soluble receptor.” Protein Sci., 10, 1955-62, (1996)) was used. The coordinates for this structure are available from the Protein Data Bank (PDB) (Bernstein et.al. J. Mol. Biol. (1977) 112 pp. 535) and electronically available via The Research Collaboratory for Structural Bioinformatics PDB at http://www.rcsb.org/pdb/ under accession code 2ILK.

It is noted that the first detected residue in the structure are Thr6 and that Thr6, Ser8, Glu9 and Asn10 have no detected side chain atoms in the structure. These residues were treated as fully exposed residues. Lys88, Arg106, Ser141 and Asp144 were reported in two different side chain conformations. For this example only the conformation labelled A was used. This structure contains coordinates for one monomer of the dimer. The coordinates of the other monomer was generated by rotation of 180 deg. around the crystallographic two-fold axis (i.e. applying the symmetry operation y, x, 1-z). All specific positions referred to herein is indicated for one monomer only.

Surface exposed atoms were identified by performing fractional ASA calculations on the IL-10 dimer molecule. The following residues were found to have more than 25% of their side chain exposed to the surface (only residues in one monomer is listed here as the dimer is completely symmetrical): S1, P2, G3, Q4, G5, T6, Q7, S8, E9, N10, S11, T13, H14, P16, G17, N18, P20, N21, R24, D25, R27, D28, S31, R32, K34, T35, Q38, M39, K40, Q42, I43, D44, N45, L46, K49, E50, S51, E54, K57, G58, S66, Q70, E74, E75, P78, Q79, N82, Q83, D84, P85, D86, K88, A89, H90, N92, S93, E96, N97, K99, T100, R102, L103, R106, R107, H109, R110, P113, K117, K119, E122, Q123, K125, N126, A127, N129, K130, Q132, E133, K134, D144, I145, N148, E151, T155, M156, K157, R159 and N160. The following residues had more than 50% of their side chain exposed to the surface: S1, P2, G3, Q4, G5, T6, Q7, S8, E9, N10, S11, T13, H14, P16, G17, P20, N21, R24, D25, D28, S31, R32, K34, T35, Q38, M39, Q42, D44, N45, K49, E50, E54, K57, G58, Q70, E74, Q79, N82, P85, D86, K88, A89, H90, S93, E96, K99, T100, L103, R107, H109, K117, K119, E122, N126, A127, N129, K130, Q132, E133, D144, N148, T155, R159 and N160.

Methods Used to Determine the In Vitro and In Vivo Activity of rhIL-10 and Variants Thereof

Primary Assay—In Vitro IL-10 Activity Assay

IL-10 can be measured by its cytokine inhibitory activity on activated peripferal blood mono-nuclear cells (PBMCs) (Science 1990; 250:830-832).

Secondary Assay—Binding Affinity of rhIL-10 or Variants Thereof to the hIL-10 Receptor

Binding of rhIL-10 or variants thereof to the hIL-10 receptor is studied using standard binding assays. The receptors may be purified extracellular receptor domains, receptors bound to purified cellular plasma membranes, or whole cells—the cellular sources being either cell lines that inherently express IL-10 receptors or cells transfected with cDNAs encoding the receptors. The ability of rhIL-10 or variants thereof to compete for the binding sites with native IL-10 is analyzed by incubating with a labeled IL-10-analog for instance biotinylated hIL-10 or radioiodinated hIL-10.

The extracellular domains of the hIL-10 receptor can optionally be coupled to Fc and immobilized in 96 well plates. RhIL-10 or variants thereof are subsequently added and the binding of these detected using either specific anti-hIL-10 antibodies or biotinylated or radioiodinated hIL-10.

Measurement of the In Vivo Half-Life of Conjugated and Unconjugated rhIL-10 and Variants Thereof

An important aspect of the invention is the prolonged biological half-life that is obtained by the conjugation of the polypeptide to the polymer moiety. The rapid decrease of hIL-10 serum concentrations has made it important to evaluate biological responses to hIL-10 treatment. Preferably, the conjugates of the present invention have prolonged serum half lifes also after i.v. administration making it possible to measure by e.g. an ELISA method or by the primary screening assay. Measurement of in vivo biological half-life can be carried out in a number of ways as described in the literature.

Measurement of the In Vivo Biological Activity of Conjugated and Unconjugated rhIL-10 and Variants Thereof

Assays to assess the in vivo biological effects of hIL-10 can be used together with the primary and secondary assays described herein to evaluate the biological efficacy of conjugated and unconjugated rhIL-10 and variants thereof.

Determination of Size Molecular Size of hIL-10 and Variants Thereof

The molecular weight of conjugated or unconjugated hIL-10 or variants thereof is determined by either SDS-PAGE, gel filtration, matrix assisted laser desorption mass spectrometry or equilibrium centrifugation

Methods for PEGylation of hIL-10 and Variants Thereof

PEGylation in Microtiter Plates of a Tagged Polypeptide with IL-10 Activity

The polypeptide exhibiting IL-10 activity is expressed with a suitable tag, e.g. any of the tags exemplified in the general description above and transferring culture broth to one or more wells in a microtiter plate capable of immobilising the tagged polypeptide. When the tag is Met-Lys-His-Gln-His-Gln-His-Gln-His-Gln-His-Gln-His-Gln-Gln, a nickel-nitrilotriacetic acid (Ni-NTA) HisSorb microtiter plate commercially available from QiaGen can be used.

After allowing for immobilising the tagged polypeptide to the microtiter plate, the wells are washed in a buffer suitable for binding and subsequent PEGylation followed by incubating the wells with the activated PEG of choice. As an example, M-SPA-5000 from Shearwater Polymers is used. The molar ratio of activated PEG to polypeptide has to be optimised, but will typically be greater than 10:1 more typically greater than 100:1. After a suitable reaction time at ambient temperature, typically around 1 hour, the reaction is stopped by removal of the activated PEG solution. The conjugated protein is eluted from the plate by incubation with a suitable buffer. Suitable elution buffers may contain Imidazole, excess NTA or another chelating compound The conjugated protein is assayed for biological activity and immunogenicity as appropriate The tag may optionally be cleaved off using a method known in the art, e.g. using diaminopeptidase and the Gin in pos-1 will be converted to pyroglutamyl with GCT (glutamylcyclotransferase) and finally cleaved off with PGAP (pyroglutamyl-aminopeptidase) giving the native protein. The process involves several steps of metal chelate affinity chromatography. Alternatively, the tagged polypeptide may be conjugated.

PEGylation of a Polypeptide Exhibiting IL-10 Activity and having a Blocked Receptor-Binding Site

In order to optimize PEGylation of hIL-10 in a manner excluding PEGylation of lysines involved in receptor recognition, the following method has been developed:

A homodimer complex consisting of a IL-10 polypeptide and the soluble domain of the IL-10 receptor in a 2:2 stoichiometry is formed in a PBS buffer at pH 7. The concentration of IL-10 polypeptide is approximately 20 ug/ml or 1 uM and the receptor is present at equimolar concentration.

M-SPA-5000 from Shearwater Polymers, Inc is added at 3 different concentration levels corresponding to 5, 20 and 100 molar excess of IL-10 polypeptide. The reaction time is 30 min at RT. After the 30 min reaction period, the pH of the reaction mixture is adjusted to pH 2.0 and the reaction mixture is applied to a Vydac C18 column and eluted with an acetonitrile gradient essentially as described (Utsumi et al, J. Biochem., vol 101, 1199-1208, (1987). Alternatively and more elegantly, an isopropanol gradient can be used.

Fractions are analyzed using the primary screening assay described herein and active PEGylated IL-10 polypeptide obtained by this method stored at −80° C. in PBS, pH 7 containing 1 mg/ml human serum albumin (HSA).

Methods of Constructing Conjugates of the Invention

Construction and Expression of IL-10 Variants

Clones comprising sequences that encode human IL-10 are deposited with the American Type Culture Collection (ATCC), Rockville, Md., under Accession Nos. 68191 and 68192.

Standard methods are used to produce transformed prokaryotic, mammalian, yeast or insect cell lines expressing large quantities of the polypeptide. Exemplary E. coli strains suitable for both expression and cloning include W3110 (ATCC Bi, 27325), X1776 (ATCC No. 31244). X2282, RR1 (ATCC Mp/31343). Exemplary mammalian cell lines include COS-7 cells, mouse L cells and CHP cells. See Sambrook (1989) and Ausubel et al., 1987 supplements).

Specifically, the method disclosed in U.S. Pat. No. 5,231,012, which is specifically and entirely incorporated herein by reference, is applied to provide recombinant IL-10.

The same method, modified according to methods well known to the person of skill in the art, enabling desired amino acid residue substitutions, is applied to produce the herein disclosed muteins of IL-10.

IL-10 and muteins thereof are purified as disclosed in U.S. Pat. No. 5,231,012 or in (Human Cytokines, Handbook of Basic and Clinical Research, Volume II, Blackwell Science, Eds. Aggarwal and Gutterman, 1996, pp. 19-42) or according to general protein purification protocols well known to the person of skill in the art.

rhIL-10 as well as all muteins of IL-10 comprising a single lysine to arginine substitution are prepared and characterized with respect to specific activity as compared to rhIL-10 to establish which, if any, lysines are critical for activity of the molecule and which may be substituted to arginine with an acceptable retention of activity.

Pegylation of IL-10 Variants

IL-10 and muteins thereof with inserted and/or deleted lysines are subjected to PEGylation by providing a surplus of SPA-PEG according to the procedure disclosed in WO 9703106 or according to the manufacturer's recommendations. Next, the specific activity of these variants is measured. The mutein(s) permitting PEGylation with retention of acceptable activity is chosen for further work.

Similarly muteins with inserted or deleted non-lysine attachment groups, e.g. aspartic or glutamic acid residues are constructed and PEGylated using a suitable PEGylation chemistry. The mutein(s) permitting PEGylation with retention of acceptable activity is chosen for further work.

The mutein(s) can be PEGylated with PEG having different molecular weights, e.g. 5, 12 or 20 kDa PEG. These molecules are controlled for continued retention of acceptable activity and subjected to characterization with respect to in vivo half-life according to the protocol of Materials and Methods. Muteins with an increased in vivo half-life are selected and exemplify the invention disclosed and claimed herein.

1. A conjugate exhibiting IL-10 activity comprising
i) a polypeptide which comprises an amino acid sequence that differs from the amino acid sequence shown in SEQ ID NO 2 in at least one amino acid residue selected from an introduced or removed amino acid residue comprising an attachment group for the non-polypeptide moiety of ii), and ii) a non-polypeptide moiety.

2. The conjugate according to claim 1, comprising 1-10 non-polypeptide moieties.

3. The conjugate according to claim 1 or 2, wherein the polypeptide i) exhibits IL-10 activity.

4. The conjugate according to any of claims 1-3, wherein said polypeptide comprises an amino acid sequence that differs from the amino acid sequence shown in SEQ ID NO 2 in the introduction of at least one lysine residue in a position selected from the group consisting of S1, P2, G3, Q4, G5, T6, Q7, S8, E9, N10, S11, T13, H14, P16, G17, N18, P20, N21, R24, D25, R27, D28, S31, R32, T35, Q38, M39, Q42, L43, D44, N45, L46, E50, S51, E54, G58, S66, Q70, E74, E75, P78, Q79, N82, Q83, D84, P85, D86, A89, H90, N92, S93, E96, N97, T100, R102, L103, R106, R107, H109, R110, P113, E122, Q123, N126, A127, N129, Q132, E133, D144, I145, N148, E151, T155, M156, R159, and N160.

5. A conjugate according to any of claims 14, wherein said polypeptide comprises an amino acid sequence that differs from the amino acid sequence shown in SEQ ID NO 2 in at least one substitution selected from the group consisting of K34X, K40X, K49X, K57X, K88X, K99X, K117X, K119X, K125X, K130X, K134X, K138X and K157X, wherein X is any amino acid residue except K and preferably is R.

6. The conjugate according to any of claims 1-5, wherein two or more residues present in SEQ ID NO 2 have been substituted with any other amino acid residue.

7. The conjugate according to any of claims 1-6, wherein the non-polypeptide moiety is a polymer molecule, e.g. PEG.

8. The conjugate according to any of claims 1-7, wherein the polypeptide is glycosylated.

9. The conjugate according to any of claims 1-8, wherein at least one glycosylation site has been introduced.

10. A conjugate according to any of claims 1-9, wherein said polypeptide comprises an amino acid sequence that differs from the amino acid sequence shown in SEQ ID NO 2 in at least one substitution selected from the group consisting of P2N+Q4S, P2N+Q4T, G3N+G5S, G3N+G5T, Q4N+T6S, Q4N, G5N+Q7S, G5N+Q7T, T6N, T6N+S8T, Q7N+E9S, Q7N+E9T, S8N+N10S, S8N+N10T, E9N, E9N+S11T, S11N+T13S, S11N, H14N+P16S, H14N+P16T, P16N+N18S, P16N+N18T, P20S, P20T, P20N+M22S, 5 P20N+M22T, L23S, L23T, R24N+L26S, R24N+L26T, D25N+R27S, D25N+R27T, R27N+A29S, R27N+A29T, D28N+F30S, D28N+F30T, S31N+V33S, S31N+V33T, R32N+K34S, R32N+K34T, K34N+F36S, K34N+F36T, T35N+F37S, T35N+F37T, Q38N+K40S, Q38N+K40T, M39N+D41S, M39N+D41T, K40N+Q42S, K40N+Q42T, Q42N+D44S, Q42N+D44T, L43N+N45S, L43N+N45T, D44N+L46S, D44N+L46T, L47S, L47T, L46N+L48S, L46N+L48T, K49N, K49N+S51T, E50N+L52S, E50N+L52T, S51N+L53S, S51N+L53T, E54N+F56S, E54N+F56T, K57N+Y59S, K57N+Y59T, G58N+L60S, G58N+L60T, S66N+M68S, S66N+M68T, Q70N+Y72S, Q70N+Y72T, E74N+V76S, E74N+V76T, P78N+A80S, P78N+A80T, Q79N+E81S, Q79N+E81T, Q83N+P85S, Q83N+P85T, P85N+187S, P85N+187T, D86N+K88S, D86N+K88T, K88N+H90S, K88N+H90T, A89N+V91S, A89N+V91T, H90N+N92S, H90N+N92T, L94S, L94T, S93N+G95S, S93N+G95T, E96N+L98S, E96N+L98T, K99S, K99T, K99N+L101S, K99N+L101T, T100SN+R102S, T100N+R102T, R102N+R104S, R102N+R104T, L103N+L 105S, L103N+L105T, R107N+H109S, R107N+H109T, H109N+F111S, H109N+F111T, P113N+E115S, P113N+E115T, K117N+K119S, K117N+K119T, E122N+V124S, E122N+V124T, Q123N+K125S, Q123N+K125T, K125N+A127S, K125N+A127T, F128S, F128T, A127N+N129S, A127N+N129T, L131S, L131T, K130N+Q132S, K130N+Q132T, Q132N+K134S, Q132N+K134T, E133N+G135S, E133N+G135T, K134N+I136S, K134N+I136T, D144N+F146S, D144N+F146T, I145N+I147S, I145N+I147T, I150S, I150T, E151N+Y153S, E151N+Y153T, T155N+K157S, T155N+K157T, M156N+I158S, M156N+I158T, K157N+R159S and K157N+R159T.

11. A conjugate according to any of claims 1-10, wherein said polypeptide comprises an amino acid sequence that differs from the amino acid sequence shown in SEQ ID NO 2 in the removal, preferably by substitution, of at least one of the amino acid residues selected from the group consisting of N116 and S118.

12. The conjugate according to any of claims 1-11, which is glycosylated and PEGylated.

13. The conjugate according to any of the preceding claims, which has a reduced susceptibility to renal clearance as compared to hIL-10.

14. The conjugate according to any of the preceding claims, which has an increased functional in vivo half-life and/or serum half-life as compared to hIL-10.

15. A substantially homogeneous preparation of a conjugate according to any of claims 1-14.

16. A polypeptide exhibiting IL-10 activity, which has the amino acid sequence of the polypeptide i) defined in any of claims 1-11.

17. A nucleotide sequence encoding a polypeptide according to claim 16.

18. An expression vector harbouring a nucleotide sequence according to claim 17.

19. A host cell comprising a nucleotide sequence according to claim 17 or an expression vector according to claim 18.

20. A method of producing a polypeptide exhibiting IL-10 activity, which method comprises subjecting the cell according to claim 19 to cultivation under conditions conducive for the expression of the polypeptide, and optionally recovering the polypeptide.

21. The method according to claim 20, which further comprises subjecting the polypeptide to conjugation to a non-polypeptide moiety.

22. A method of increasing the functional in vivo half-life and/or serum half-life of a polypeptide exhibiting IL-10 activity, which method comprises introducing an amino acid residue change as defined in any of claims 1-11 and subjecting the resulting modified polypeptide to conjugation with the appropriate non-polypeptide moiety.

23. A method for preparing a conjugate according to any of claims 1-14, wherein the polypeptide i) is allowed to react with the non-polypeptide moiety ii) under conditions conducive for the conjugation to take place, and the resulting conjugate is recovered.

24. The method according to any of claims 21-23, wherein the conjugation to the non-polypeptide moiety is conducted in the presence of a molar excess of the non-polypeptide moiety whereby a substantially homogenous conjugate preparation is obtained.

25. A pharmaceutical composition comprising a) a conjugate according to any of claims 1-14 or a preparation according to claim 15 and b) a pharmaceutically acceptable diluent, carrier or adjuvant.

26. A conjugate according to any of claims 1-14, a preparation according to claim 15, or a composition according to claim 25 for use in the treatment of diseases, in particular treatment of inflammatory diseases, such as rheumatoid arthritis, and in connection with transplantation, immunodeficiencies and parasitic infections.

27. Use of a conjugate according to any of claims 1-14, a preparation according to claim 15, or a composition according to claim 25 for the treatment of diseases, in particular treatment of inflammatory diseases, such as rheumatoid arthritis, and in connection with transplantation, immunodeficiencies and parasitic infections.

28. Use of a conjugate according to any of claims 1-14, a preparation according to claim 15, or a composition according to claim 25 for the manufacture of a medicament for treatment of diseases, in particular treatment of inflammatory diseases, such as rheumatoid arthritis, and in connection with transplantation, immunodeficiencies and parasitic infections.

29. A method of treating a mammal having an inflammatory diseases, such as rheumatoid arthritis, and in connection with transplantation, immunodeficiencies and parasitic infections, comprising administering to a mammal in need thereof an effective amount of a conjugate according to any of claims 1-14, a preparation according to claim 15, or a composition according to claim 25.
